SEQUENCE LISTING (5 sequences)

SEQ ID NO: 1 - protein, 20 aa, Artificial Sequence
  Description of Artificial Sequence Ligase nucleic acid precursor molecule
YSVTVKWGKEKFEGVELNTC

SEQ ID NO: 2 - protein, 18 aa, Artificial Sequence
  Description of Artificial Sequence Synthetic Peptide
CKSLIDQYFGVEFETTMK

SEQ ID NO: 3 - protein, 21 aa, Artificial Sequence
  Description of Artificial Sequence Synthetic Peptide
CKLRLQEEITKQSPTLQRNAL

SEQ ID NO: 4 - protein, 21 aa, Artificial Sequence
  Description of Artificial Sequence Synthetic Peptide
CTPELQEKMVSFRSKFKDLED

SEQ ID NO: 5 - protein, 275 aa, Homo sapiens
GCLKMAAEEPQQQKQEPLGSDSEGVNCLAYDEAIMAQQDRIQQEIAVQNPLVSERLELSVLYKEYAEDDNIYQQKIKDLHKKYSYIRKTRPDGNCFYRAFGFSHLEALLDDSKELQRFKAVSAKSKEDLVSQGFTEFTIEDFHNTFMDLIEQVEKQTSVADLLASFNDQSTSDYLVVYLRLLTSGYLQRESKFFEHFIEGGRTVKEFCQQEVEPMCKESDHIHIIALAQALSVSIQVEYMDRGEGGTTNPHIFPEGSEPKVYLLYRPGHYDILYK

(19) United States
(12) Patent Application Publication
(10) Pub. No.: US 2005/0277762 A1
(43) Pub. Date: Dec. 15, 2005

(54) Semisynthetic protein-based site-directed probes for identification and inhibition of active sites, and methods therefor

(76) Inventors: Hidde L. Ploegh, Brookline, MA (US); Huib Ovaa, Boston, MA (US); Anna Borodovsky, Somerville, MA (US); Benedikt Kessler, Cambridge, MA (US); Joris Hemelaar, Boston, MA (US); Paul Galardy, North Andover, MA (US); Nagamalleswari Kolli, Doraville, GA (US); Tudevin Gan-Erdene, Atlanta, GA (US); Keith D. Wilkinson, Lilburn, GA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/423,421

(22) Filed: Apr. 25, 2003

Related U.S. Application Data
(60) Provisional application No. 60/375,586, filed on Apr. 25, 2002.

(51) Int. Cl. C07K 14/00

(52) U.S. Cl. 530/350

(57) ABSTRACT

A C-terminally modified Ubiquitin (Ub) derivative) ubiquitin vinyl sulfone (UbVS), which is specific for deubiquitinating enzymes (DUBs), was synthesized as an active site directed probe that irreversibly modifies a subset of Ub C-terminal hydrolases (UCHs) and Ub specific processing proteases (UBPs), is provided. [¹²⁵I]-UbVS modifies 6 of the 17 known and putative yeast deubiquitinating enzymes, namely Yuh1p, Ubp1p, Ubp2p, Ubp6p, Ubp12p and Ubp15p. In mammalian cells, a greater number of polypeptides is labeled, most of which are DUBs. An additional DUB that associates with the mammalian 26S proteasome, novel protein USP14, a mammalian homolog of yeast Ubp6p that is bound to the proteasome, is provided.

### RELATED APPLICATION

This application claims the benefit of provisional application Ser. No. 60/375,586 filed Apr. 25, 2002, and which is incorporated herein by reference.

### GOVERNMENT RIGHTS

This invention was made in part with government support under grant numbers GM062502, GM30308, and GM66355 awarded by the National Institutes of Health. The Ad government has certain rights in the invention.

### TECHNICAL FIELD

A method for identifying and designing site directed inhibitors for proteomic sets, such as proteins that are enzymes involved in metabolism of ubiquitin and ubiquitin-like proteins of the cell, is provided, as are methods for identification and modulation of enzymatic pathways, for treatment of disorders associated with this pathway, and compositions for treatment of these disorders, and compositions for investigation of the biological function of a pathway in a proteomic set.

### BACKGROUND

It is desirable to have chemical tools for analysis of the proteome, the tools being based on a shared in vivo function. An example of such a function is covalent modification of proteins by ubiquitin (Ub), a small 76 amino acid protein, which is important in the control of many cellular processes (Hershko et al., Annu Rev Biochem, 67, 425-79, 1998). Ubiquitination primarily serves as a targeting signal, and proteins carrying the most common type of poly-Ub chain are targeted for destruction by the ubiquitin-proteasome pathway, responsible for the majority of cytosolic proteolysis (Ciechanover et al., Proc Natl Acad Sci USA, 95, 2727-30, 1998). Ub is attached to proteins through an isopeptide linkage, involving the C-terminal carboxylate of Ub and the ε-NH₂ of a lysine sidechain, (Ciechanover et al., Mol Biol Rep, 26, 59-64, 1999; Hodgins et al., J. Biol. Chem., 271, 28766-28771, 1996). The enzyme cascade involved in Ub-conjugation and poly-Ub chain formation comprises at least three distinct sets of enzymatic activities including the Ub-activating enzyme E1, Ub-conjugating enzymes (E2) and E3 ligases (reviewed in Hershko and Ciechanover, Annu Rev Biochem, 67, 425-79, 1998). Ubiquitin-dependent events may also be controlled at the level of deubiquitination (Wilkinson, FASEB J, 11, 1245-56, 1997). Removal of Ub is carried out by deubiquitinating enzymes (DUBs), a large family of proteases that can release poly-Ub chains from proteins to be degraded by the 26S proteasome, recycle monomeric Ub, liberate Ub from the Ub-fusion protein precursors, reverse regulatory ubiquitination and edit inappropriately ubiquitinated proteins (reviewed in Chung et al., Biochem Biophys Res Commun, 266, 63340, 1999).

DUBs can be subdivided into Ub C-terminal hydrolases (UCHs) and Ub-specific processing proteases (UBPs). In vitro, UBPs hydrolyze isopeptide bonds between Ub and folded protein domains, such as additional Ub moieties or target proteins. Thus, UBPs exhibit broad substrate specificity (Wilkinson, FASEB J, 11, 1245-56, 1997). UCHs generally cleave bonds between Ub and an unfolded polypeptide or Ub and small substituents (Pickart et al., J Biol Chem, 260, 7903-10, 1985; Wilkinson, FASEB J, 11, 1245-56, 1997; Wilkinson et al., Biochemistry, 25, 6644-9, 1986). Deletion studies in yeast suggest that the substrate specificities of UCHs and UBPs overlap (Amerik et al., Biol Chem, 381, 981-92, 2000; Baker et al., J Biol Chem, 267, 23364-75,1992). Both UBPs and UCHs can associate with the 26S proteasome and are involved in the regulation of Ub-dependent proteolysis (Voges et al., Annu. Rev. Biochem, 68, 1999).

A UCH enzyme within the 19S regulatory complex has been reported in mammals (UCH37; Lam et al., J Biol Chem, 272, 2843846,1997), Drosophila (p37a; Holzl et al., J Cell Biol, 150, 119-130, 2000), and S. pombe (Uch2p; Li et al., Biochem Biophys Res Commun, 272, 270-5, 2900) and is thought to edit Ub-chains on protein substrates before they are degraded (Lam et al., J Biol Chem, 272, 28438-46, 1997; Lam et al., Nature, 385, 737-40, 1997). Doa4 in yeast and Ap-UCH in Aplysia are more transiently associated with the proteolytic complex and may cleave Ub from remnants of degraded proteins (Hegde et al., Cell, 89, 115-26, 1997; Papa et al., Mol Biol Cell, 10, 741-56, 1999; Papa et al., Nature, 366, 313-9, 1993). In higher organisms, UCHs are expressed in a tissue specific manner and are linked to a variety of cellular functions and diseases, such as Parkinson's disease (UCH-L1; Wilkinson et al., Biochem Soc Trans, 20, 631-7, 1992), the function of BRCA1 (BAP-1; Jensen et al., Oncogene, 16, 1097-112, 1998), and long-term nerve potentiation in Aplysia (Ap-UCH; Hegde et al., Cell, 89, 115-26,1997). UBPs also play a role in determination of cell fate (fat facets; Huang et al., Science, 270, 1828-31, 1995), transcriptional silencing (Ubp3; Moazed et al., Cell, 86, 667-77, 1996), response to cytokines (DUB1 and 2; Zhu et al., Mol Cell Biol, 16,4808-17, 1996) and oncogenic transformation (tre-2; Papa et al., Nature, 366, 313-9, 1993).

The yeast genome encodes 17 DUBs (UBPs/UCHs), however, the number of UBPs and UCHs in mammals is likely to be far greater (Chung and Baek, Biochem Biophys Res Commun, 266, 63340, 1999). Enzymatic activity has been demonstrated for many DUBs using model substrates (Amerik et al., Biol Chem, 381, 981-92, 2000), and for others an assignment was made based on homology with known family members (Wilkinson, FASEB J, 11, 1245-56, 1997). The multiplicity of DUBs raises the possibility of distinct functions and/or protein substrates for each DUB.

The Ub pathway has been shown to play an important regulatory role in processes such as cell cycle control, signal transduction and the immune response, and has been implicated in the development of cancers and neurodegenerative diseases. The Ubl proteins have been implicated in a variety of cellular processes such as autophagy, interferon response, nuclear translocation, cell cycle progression and apoptosis. Table 1 shows relationships known between the Ub pathway and various pathological conditions.

### SUMMARY

The multiplicity of enzymes that carry out enzymatic reactions on ubiquitin and Ubl proteins makes design of tools to study them highly desirable. As these enzyme systems are known to play a role in many pathological conditions, tools to study these enzymes have potential applications in diagnosis and/or treatment.

Chemoselective reaction tools for elucidation of enzymatic specificies of DUBs and relationships of these and related ubiquitin and ubiquitin-related proteins to various disease states will provide new targets for drug design. Accordingly, the invention in one embodiment features a fusion peptide comprising an amino acid sequence of components, in order from the amino terminus: an epitope label; an amino acid sequence of a ubiquitin protein or a ubiquitin-like protein; an intein; and a chitin binding protein. A “ubiquitin protein” as used herein and in the claims means ubiquitin and any related proteins or fragments thereof that share the same affinity and enzyme activities of ubiquitin, including any affinities and activities within a 10-fold range of that of ubiquitin. A related embodiment is a vector encoding the fusion peptide. The epitope label can be replaced with other means of identification or tagging such as a biotin. In alternative embodiments, the ubiquitin protein can be a ubiquitin-like protein, for example, can be selected from the group consisting of UCH-L3, APG8, APG12, UCRP, SUMO-1, NEDD8, HUB1, URM-1, FAT10 and Fau. A related embodiment is a nucleic acid encoding the fusion peptide, for example; a vector encoding the fusion peptide.

In another related embodiment, a semi-synthetic protein-based site directed probe for identification and inhibition of a class of genomic proteins, the probe comprising the epitope label and ubiquitin protein of the peptide is provided, the probe further comprising a potential inhibitory group at the carboxy terminus of the ubiquitin protein wherein the inhibitory group is specific for an enzymatic acitvity of a ubiquinating or a deubiquinating enzyme. The site-directed probe can be reversibly inhibitory, for example, the reversible inhibitory group is an aldehyde or a boronate. Alternatively, the group is irreversibly inhibitory, for example, the irreversibly inhibitory group is an alkylating agent, an electron withdrawing group. The irreversibly inhibitory group can be Michael acceptor or an alkylating group. For example, the Michael acceptor is selected from compounds such as 3-vinylmethylsulfone; 3-vinylphenylsulfone; 3-vinylnitrile; and 2 carboxyvinylmethane and derivatives of these and other similar corestructures.

Another embodiment of the invention is a method of obtaining a semi-synthetic protein-based site directed probe for identification and inhibition of a subset of a proteome, the method comprising:

providing a fusion protein encoded by a nucleic acid vector, the fusion protein having: an epitope tag, a domain having an amino acid sequence from a member protein of the subset, an intein, and an affinity creating binding peptide;

breaking a peptide bond located between the domain and the intein, to yield a sulfoxide thioester at the carboxy terminus of the representative peptide; and

further reacting the sulfoxide thioester to yield anactive reversible aldehyde or an electron withdrawing group at the carboxy terminus of the domain, thereby obtaining the site directed probe. For example, the member protein is a ubiquitin protein or a ubiquitin-like protein. The member protein is selected from the group consisting of UCH-L3, APG8, APG12, UCRP, SUMO-1, NEDD-8, HUB1, URM-1, FAT10 and Fau. Further, the epitope tag is hemagglutinin, Flag, Myc, or His6. Other types of tags are possible, for example, biotin-binding groups such as streptavidin, and HIV TAT protein which is useful for rendering the protein cell-permeable, and TEV-cleavable domains.

A further embodiment of the method is provided, in which prior to breaking the peptide bond between the intein and the domain, the method further comprises purifying the fusion protein by contacting a preparation comprising the fusion protein the with an immobilized binding partner of the affinity binding peptide. In some embodiments, the affinity binding domain is a chitin binding domain, and the immobilized binding partner is immobilized chitin. The invention in a related embodiment provides the probe identified by the method.

In another embodiment, the invention provides a method for obtaining a semi-synthetic protein-based site directed probe for identification and inhibition of ubiquitin and ubiquitin-like proteins, the method comprising:

providing a fusion protein encoded by a nucleic acid vector, the fusion protein having: an hemagglutinin tag, a domain from a ubiquitin or a ubiquitin-like protein, an intein, and a chitin binding peptide;

breaking a peptide bond located between the domain and the intein, to yield a thioester at the carboxy terminus of the representative; and

further reacting the thioester to yield an active reversible aldehyde, alkylating moiety or an electron withdrawing group at the carboxyterminus of the domain, thereby obtaining the site directed probe.

In yet another embodiment, the invention provides a method of identifying a subset of a proteome, wherein members of the subset share a functional pathway, the method comprising:

preparing a semi-synthetic protein-based site directed probe having an amino acid sequence comprising an epitope tag, a peptide from a member of the subset having an enzyme activity, and an inhibitory group at the carboxy terminus of the member peptide, the inhibitory group having ability to contact and inhibit an activity of the enzyme;

contacting a lysate of the cell with the probe; and

analyzing the lysate by reducing SDS gel electrophesis and immunoblotting with an antibody specific for the tag, such that cell lysate components encoded by the members of the subset bind to the probe, and react with the antibody to visualize bands on the gel, thereby identifying the subset of the proteome that share the pathway. For example, the pathway is ubiquitination or deubinquitination. In a further embodiment after the analyzing step, the subset can be immunoisolated by performing mass spectrometry.

Further provided are nucleic acid sequences encoding any of the vectors provided herein. A protein identified according to the method is also provided. A protein is provided having an amino acid sequence comprising, using the one letter code for amino acids, GCLKMAAEEPQQQKQEPLGSDSEGVNCLAYDEAIMAQQDRIQQEIAVQNPL VSERLELSVLYKEYAEDDNIYQQKIKDLHKKYSYIRKTRPDGNCFYRAFGFS HLEALLDDSKELQRFKAVSAKSKEDLVSQGFTEFTIEDFHNTFMDLIEQVEK QTSVADLLASFNDQSTSDYLVVYLRLLTSGYLQRESKFFEHFIEGGRTVKEF CQQEVEPMCKESDHIHIIALAQALSVSIQVEYMDRGEGGTTNPHIFPEGSEP KVYLLYRPGHYDILYK.

An embodiment of the invention is a kit for identifying ubiquitination and deubiquitination proteins in a cell, comprising a semi-synthetic protein-based site directed probe having an epitope tag, an amino acid sequence of a ubiquitin or ubiquitin-like enzyme, and an inhibitory compound covalently bound to the carboxy terminus of the amino acid sequence, and instructions for use. For example, a protein having an amino acid sequence according to SEQ ID NO:5 is provided.

### BRIEF DESCRIPTION OF THE DRAWINGS

FIG. 1A is sketch of chemical reactions for synthesis of UbVS C-terminal modification of ubiquitin, compound 1, to generate ubiquitin vinyl sulfone, compound 6. An amount of 25 mg of compound 1 was converted to ubiquitin-75 ethyl ester, compound 2, by treatment with 2.5 mg trypsin in presence of 1.6 M glycine ethyl ester (GlyOEt) and 20% PEG 20,000. Compound 2 was treated with hydrazine monohydrate and HCl to generate ubiquitin-75 hydrazine, compound 3, which was dialyzed against water and converted to ubiquitin-75 azide, compound 4, by treatment with 0.5 M nitrous acid for 1 min at −5° C. Compound 4 was immediately reacted with the TFA salt of glycine vinyl sulfone, compound 5, in presence of TEA, generating compound 6. FIG. 1B is a readout of purified UbVS being resolved on an analytical C4 column using a 0.l% formic acid/acetonitrile buffer system. Eluate was analyzed by on-line ES-MS. The indicated multicharged species correspond to a molecular weight of 8624.9 Da, in agreement with the predicted MW of UbVS (8625 Da). FIG. 1C is a photograph of an SDS-PAGE characterization of a reaction in which recombinant purified UCH-L3 was incubated with a substoichiometric amount of [¹²⁵I]-UbVS in PBS for 45 min at 37° C., with or without pre-treatment with 2 mM N-ethylmaleimide. Protein conjugates were resolved by 12.5% SDS-PAGE under reducing conditions, and visualized by silver stain (left panel) and autoradiography (right panel).

FIG. 2A is a photograph of SDS-PAGE characterization of [¹²⁵I]-UbVS specifically labeling a subset of yeast DUBs from 100 μg of post-nuclear lysates from DUB deletion strains that were incubated with 1×10⁶ cpm of [¹²⁵I]-UbVS for 45 min at 37° C. Reactions were quenched with sample buffer, resolved by 10% SDS-PAGE and analyzed by autoradiography. Identity of bands was assigned based on the absence of a band corresponding to the molecular weight of a DUB deleted in that strain. FIG. 2B is a photograph of SDS-PAGE characterization showing lysates from wild type yeast that were pre-incubated with increasing concentrations of Ubal competitor for 30 min at room temperature, prior to addition of [¹²⁵I]-UbVS and SDS-PAGE as described in FIG. 2A.

FIG. 3A is a photograph of SDS-PAGE characterization of [¹²⁵I]-UbVS labeling mammalian DUBs from single cell suspensions prepared from tissues of a male B6 mouse: muscle (MU), brain (BR), kidney (KI), thymus (TH), and spleen (SP). Lysate (50 μg) was treated with 1×10⁶ cpm [¹²⁵I]-UbVS as described in FIG. 2A, and resolved by 10% SDS-PAGE. FIG. 3B is a photograph of SDS-PAGE characterization of 20 μg of post-nuclear lysates from NIH 3T3 cells that were pre-treated with increasing concentrations of Ubal as competitor for 30 min at 37° C., followed by labeling with [¹²⁵I]-UbVS. FIG. 3C is a photograph of SDS-PAGE characterization of 50 μg of lysates from EL-4 cells that were treated with 2 μM UbVS at 37° C. for 1 hr, and resolved by 10% SDS-PAGE and immunoblotted with the r201 rabbit anti-serum against USP7.

FIG. 4A is a photograph of SDS-PAGE characterization of USP14 associating with the 26S proteasome, using EL-4 cell lysates fractionated on a Superose 6 FPLC column to isolate high molecular weight complexes, as described in Materials and methods. Fractions were labeled with 0.5×10⁶ cpm of[¹²⁵I]-UbVS and resolved by SDS-PAGE. FIG. 4B is a photograph of SDS-PAGE characterization of 80 μg of EL4 cell lysates treated with 2×10⁶ cpm [¹²⁵I]-UbVS and immunoprecipitated with anti-20S proteasome anti-serum using proteasome IP buffer or denatured with 1% SDS and immunoprecipitated with anti-USP14 antiserum HM433 using NET buffer. FIG. 4C is a photograph of SDS-PAGE characterization of EL4 lysates fractionated on a Superose 6 column as described in FIG. 4A. A volume of 1 ml of each fraction was incubated with [¹²⁵I]-UbVS and immunoprecipitated for the proteasome as in B (top panel). A volume of 50 μl of each fraction was analyzed for presence of proteasome subunits by immunoblot with indicated antibodies against components of the 20S and the 19S (lower panels).

FIG. 5A is a photograph of SDS-PAGE characterization of ¹²⁵I]-UbVS labeling of proteasome-associated USP14, which is shown to be increased upon proteasome inhibition, using 5×10⁶ EL4 cells treated with 50 μM NLVS for indicated times. Lysates were normalized for total protein and incubated with [¹²⁵I]-UbVS or [¹²⁵I]-NLVS for 1 hr. Proteasomes were immunoprecipitated as described in FIG. 4B. FIG. 5B is a bar graph that shows intensities of USP14 bands, quantified by densitometry, in untreated (black) and NLVS-treated (gray) cells. FIG. 5C is a photograph of SDS-PAGE characterization of EL4 cells that were incubated with 50 μM NLVS, or ZL₃VS, or 4 μM epoxomicin for the indicated times. Proteasomes were immunoprecipitated as described in FIG. 4B.

FIG. 6A is a photograph of SDS-PAGE characterization showing that activity of USP14 is increased in response to proteasome inhibition, with subcellular fractions from EL-4 cells previously treated with 50 μM NLVS for 5 hours, incubated with [¹²⁵I]-UbVS, resolved by 10% SDS-PAGE, and visualized by autoradiography. The term 1hS means 1 hour supernatant, 5 hS means 5 hour supernatant, 5 hP means 5 hour pellet. FIG. 6B is a photograph of SDS-PAGE characterization of parallel samples resolved by 10% SDS-PAGE and inmnunoblotted with anti-USP14 and anti-Mss1 anti-sera. FIG. 6C is a bar graph that shows subcellular fractions prepared from EL4 cell extracts that were incubated with 50 μM NLVS for 3 hours, resolved by 10% SDS-PAGE, and visualized by autoradiography (upper panel), and by immunoblot using anti-USP14 (lower panel). Intensities of USP14 bands obtained from four independent experiments were quantified by densitometry and normalized to the amount of USP14 labeled by ¹²⁵I]-UbVS (upper panel) and USP14 protein (lower panel) observed in 1 hour supernatant fractions.

FIG. 7 is a flow chart showing bacterial production of a fusion protein from an intein vector encoding a fusion protein having, from the direction of the amino terminus to the caarboxy terminus, an epitope tag such as hemagglutinin (HA), a peptide which is ubiquitin or a ubiquitin-like protein lacking the C-terminal amino acid, an intein, and a peptide capable of binding to an affinity material such as a chitin binding domain. Expression of the fusion protein in a microbial cell is regulated by the operon repressor LacZ, e.g., so that expression is induced by addition of IPTG. The protein is purified by a single step of affinity chromatography or batch purification on the basis of affinity to chitin immobilized on the column or on a suspension of beads. The protein is cleaved by treatment with the reagent mercapto ethane sulfonic acid sodium salt (MESNA), to release the ubiquitin or ubiquitin-like peptide portion as a thioester rather than having a C-terminal amino acid; the intein-containing portion is retained on the column or bead. As a result of an intramolecular rearrangement, the peptide at the carboxy terminus has an amine linked to a chemical group, R.

FIG. 8A is a drawing that shows the “new route” for synthesis of potential inhibitors, which comprises introduction of “warheads” following a series of chemical steps after cleavage with MESNA. Use of 1-amino-2,2-dimethoxyalkane followed by acid catalyzed hydrolysis of the resulting acetal leads to introduction of an aldehyde, which is a reversible inhibitor of a variety of ubiquitin-related enzymes. A further Wittig reaction with stabilized ylides of the obtained aldehydes in aqueous solution leads directly to peptide-michael acceptors having an electron withdrawing group (EWG), which can be an active irreversible Michael-acceptor inhibitor. FIG. 8B is a drawing that shows the Wittig reaction performed on an intact protein. The Wittig reaction allows for convenient and rapid introduction of variation in shape, electrophilicity and position of the electrophilic trap.

FIG. 9 is a drawing that shows the chemical structures of a series of inhibitors at the carboxy terminus of ubiquitin-gly-75, synthesized by the initial route (left) involving direct chemical ligation of an electrophile at the thioester and by the new route (right) involving a wittig reaction on the corresponding aldehyde.

FIG. 10A is a drawing showing semi-synthetic construction of the HAUb vectors, which are constructs encoding HA-Ub peptide fusions with C-terminal modifications, the drawing showing a choice of several different reactive groups. FIG. 10B shows the chemical structures of a series of reactive groups. FIG. 10C is a photograph of an SDS-PAGE analysis of an anti-hemagglutin (anti-HA) blot antibody blot (Western) that shows protein bands fractionated on the basis of molecular weight which are labeled as shown in FIG. 4 by a series of vectors derived from vector HAUb. A sample of 20 μl of EL4 cell lysate was incubated with 0.5 μM of each of vectors HAUb (control without a reactive group), HAUBVS, HAUbVMe, HAUbVSPh, and HAUbBr1, HAUbBr2, or 1 μM of each of HAUbCN, HAUbC1, and HAUbBr3. Proteins labeled with the vectors were resolved by 8% reducing SDS-PAGE and immunoblotted with anti-HA antibody to reveal the bands shown in each lane. Data show that different HAUb-derived active site-directed probes show distinct labeling profiles.

FIG. 11 is a photograph of an electrophoretogram of proteins bound by different reactive site inhibitors using the HAUb vectors indicated in each lane. The proteins were immunoisolated using antibody specific for the HA epitope tag, were fractionated by electrophoresis performed under non-denaturing conditions, and were visualized with silver stain so that all proteins present are shown. Open circles indicate 19S cap bound USPs and open circles the 19S cap subunits.

FIG. 12 is a family tree drawing of the relationships of sequences of the UBP family of enzymes. The catalytic domains of the UBPs annotated in SwissProt or GenBank databases or sequences herein were assigned based on ProSite parameters. Catalytic domains werw aligned using MegAlign program of the DNAStar software packing (using a Clustal V algorithm PAM250 Matrix); all catalytic residue signatures were well aligned, except CYLD1, for which a recognizable His box could not be found. A neighbor-joining phylogenetic tree was generated based on alignment. Shaded boxes indicate enzymes targeted by HAUb probes herein (HAUbVS, HAUbVME and HAUbBr2) that were identified by mass spectroscopy.

FIG. 13 is a set of photographs of gel electrophoretograms showing the Ub1-Vs probes label distinct sets of proteins in EL-4 lysates. FIG. 13A shows use of ¹²⁵[I]-Ub-VS. FIG. 13B shows use of ¹²⁵[I]-Nedd8-VS. FIG. 13C shows use of ¹²⁵[I]-UCRP-VS. FIG. 13D shows use of ¹²⁵[I]-SUMO-1-VS. Vinyl sulfone derivatives of each Ubl (ubiquitin-like proteins) were radiolabeled with Na¹²⁵I and were incubated with EL-4 cell lysates. In each sample, 5×10⁵ cpm of ¹²⁵[I]-labeled probe and 40μg of EL-4 lysate were used. The left-most lane in each Fig. shows electrophoresis with no lysate added (probe alone control); remaining lanes show contents of EL-4 sample pretreatment as indicated on the top: EL-4 alone (no pretreatment), or pretreatment with 1 mM PMSF, 10 mM NEM, or 20 mM NEM, respectively, prior to addition of probe. Data show that the different Ubl probes have different profiles of protein interactions in the same cell type.

FIG. 14 is a photograph of gel electrophoretogram of sets of different lymphocytic cell lines (LCLs) probed with HAUb probe HAUbVME. The lanes labeled LCLs are Epstein-Barr virus infected B-cell lines. Labeled bands were visualized with anti-HA immunoblot.

### DETAILED DESCRIPTION OF EMBODIMENTS

Synthesis of UbVS (ubiquitin vinyl sulfone), a covalent inhibitor of deubiquitinating enzymes (DUBs), and other similar probes is provided. Genetic criteria as well as a comparison with a reversible inhibitor (Ubal) both yield to information that establishes specificity of the affinity probe. UbVS was used to detect DUBs in crude extracts and to uncover the presence of an additional DUB, USP14 in association with the 26S proteasome. Surprisingly, the intensity of USP14 labeling with UbVS is inversely proportional to the activity of the proteasome, thus demonstrating physical and functional interaction among different components of the ubiquitin-proteasome pathway.

Known UBPs and UCHs are thiol proteases with specificity for Ub. Introduction of a suitable electrophile at the C-terminus of Ub might allow irreversible trapping of UBPs and UCHs as covalent adducts. By labeling such probes with ¹²⁵I, it should be possible to directly visualize active DUBs and other proteins involved in ubiquitination and deubiquitination.

Vinyl sulfones are versatile functional groups ideally suited to inhibit thiol proteases (Palmer et al., J Med Chem, 38, 3193-6, 1995). Their reactivity is not necessarily limited to thiol proteases, as proteasomes, N-terminal threonine hydrolases, also are efficiently inhibited by peptide vinyl sulfones (Bogyo et al., Proc Natl Acad Sci USA, 94,6629-34, 1997). Using the well-established method of trypsin-catalyzed transpeptidation to modify ubiquitin (Ub) (Wilkinson et al., Biochemistry, 25, 6644-9, 1986), the C-terminally modified vinyl sulfone derivative of Ub, hereinafter referred to as UbVS, was synthesized as a tool to obtain specificity of protein labeling The use of [¹²⁵I]-UbVS allows the visualization of active UBPs/UCHs (UB-specific processing proteases/Ub c-terminal hydrolases) in crude cell extracts. In addition to the previously characterized 37 kDa enzyme (Lam et al., J. Biol Chem, 272, 28438-46, 1997), a novel second DUB is found herein to be associated with the mammalian proteasome. This enzyme is identified as USP14, a mammalian homolog of yeast Ubp6. USP14 associates with the 26S complex and its activity, unlike that of p37, is affected by proteasome inhibition both in vitro and in living cells.

A key advantage of using [¹²⁵I]-UbVS as a probe for UBPs and UCHs is that this compound has a mechanism-based, irreversible mode of labeling conferred by the vinyl sulfone moiety (Palmer et al., J Med Chem, 38, 3193-6, 1995). The thioether linkage results from attack of the DUB active site thiol on the vinyl sulfone of UbVS, a reactive Michael acceptor. This type of linkage is resistant to the reducing sample buffers used in SDS-PAGE, unlike the adduct formed with the Ub-isonitrile derivative described by Lam and co-workers (Lam et al., Nature, 385, 73740, 1997). It is therefore possible to contact [¹²⁵I]-UbVS with crude lysates, thereby enabling direct visualization of a subset of the active UBPs and UCHs. The intensity of labeling of a given enzyme in the cell lysate by [¹²⁵I]-bVS then corresponds to its activity. The use of UbVS obviates the need for purification of DUBs of interest in order to assess their activities.

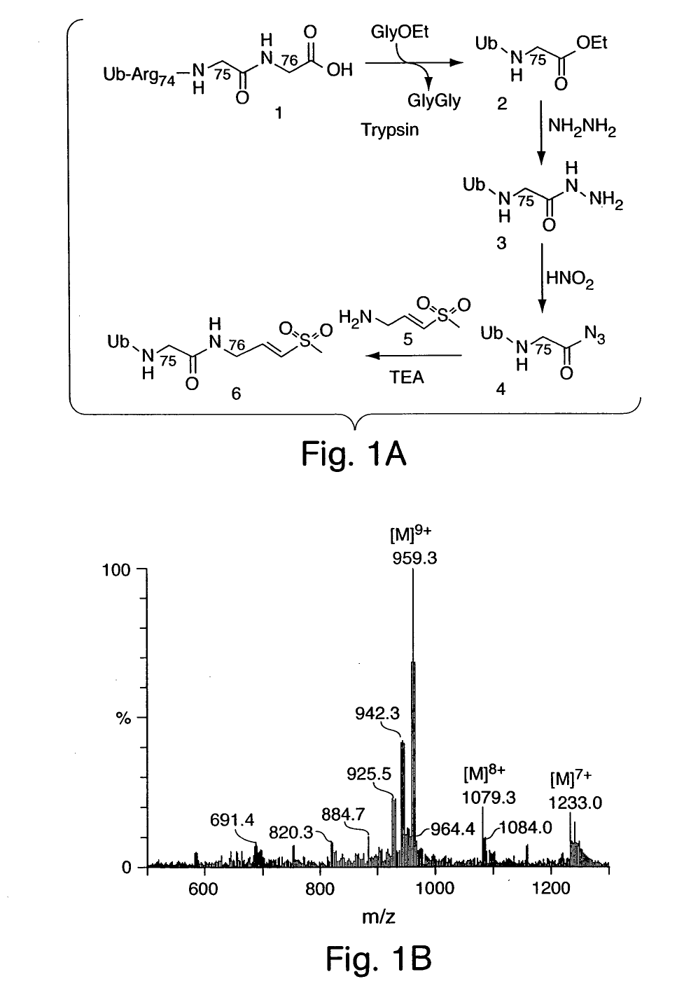

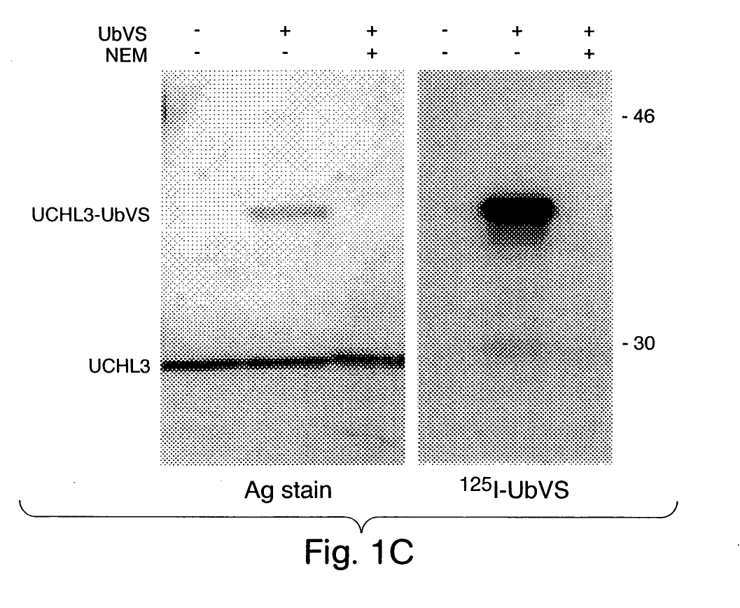

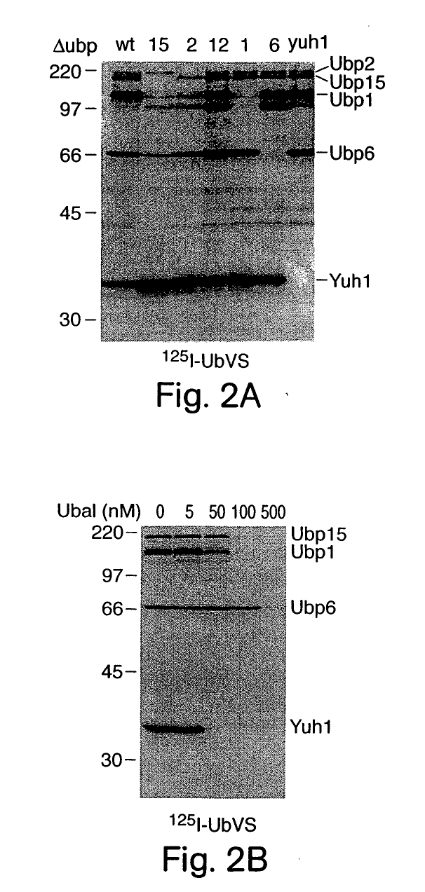

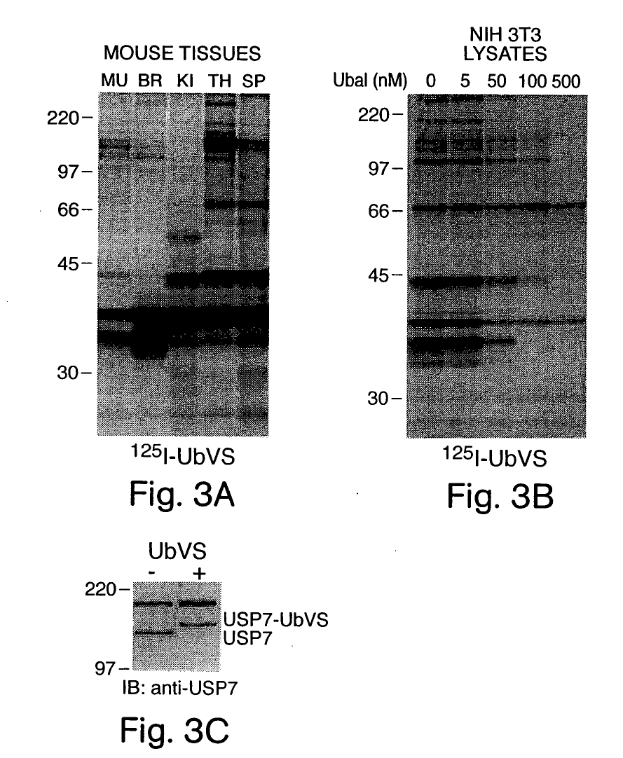

Prior treatment of the recombinant UCH-L3 enzyme or crude extracts with an alkylating agent, N-ethylmaleimide, abolishes labeling (FIG. 1). This shows the presence of an active site cysteine involved in catalytic activity of UCH-L3 as exemplary of DUBs (Wilkinson, FASEB J, 11, 1245-56, 1997). Ubal, a known inhibitor of this class of enzymes, is an effective competitor of [¹²⁵I]-UbVS labeling of DUB in cell extracts (FIG. 2B and 3B). Both Ubal and UbVS bind UCH-L3 with a sub-micromolar binding constant (Dang et al., Biochemistry, 37, 1868-79, 1998), which is a range suggesting that such an inhibitor might be useful as a lead compound for a pharmaceutical such as a therapeutic agent.

Specific labeling by [¹²⁵I]-UbVS is based also on genetic data. Deletion mutants of DUB genes in budding yeast lack [¹²⁵I]-UbVS labeling of polypeptides of the molecular weight expected for the particular deletion. In fact, 6 of 17 known or putative DUBs account for all of the bands labeled here in crude yeast extracts, demonstrating specificity of UbVS for this class of enzymes. The multiple bands seen for Ubp1p (FIG. 2A) are likely due to proteolytic cleavage. Not all yeast DUBs are labeled by [¹²⁵I]-UbVS; without being bound by any particular mechanism, this observation can be explained in several ways. The vinyl sulfone substitution may hinder interaction with a particular UBP's active site and thereby render that UBP refractory to labeling. Further, several UBPs may have higher affinity for poly-Ub chains than for monomeric Ub, as seen for Isopeptidase T (Wilkinson et al., Biochemist, 34,14535-46, 1995). Finally, expression levels of some UBPs in logarithmically growing or stationary phase yeast may be too low for detection by this method. Inspection of the mRNA levels for the DUB family based on yeast genomic array analysis shows that 3 of the labeled DUBs (Ubp1, 2 and 6) have higher mRNA transcript levels then the majority of other DUBs, suggesting that increased expression may reveal other DUBs capable of detection by labeling (Holstege et al., Cell, 95, 717-28, 1998).

Yeast UBPs are generally considered to be non-essential, as genetic deletion of single or multiple DUBs can be performed without conferring a lethal or severe phenotype (Amerik et al., Biol Chem, 381, 981-92, 2000; Baker et al., J Biol Chem, 267, 23364-75, 1992). Without being bound by any particular mechanism, results with yeast extracts presented herein show that functions of many UBPs can overlap, or that functions can be regulatory and restricted to particular substrates or pathways, or that not every enzyme is obligatory for removal of Ub from substrates prior to proteasomal proteolysis. Use is made herein of the yeast system to identify the specificity and activity of the HAUb probes. Synthesis of Ub derivatives with electrophiles other than vinyl sulfone can identify additional active members of the DUB family.

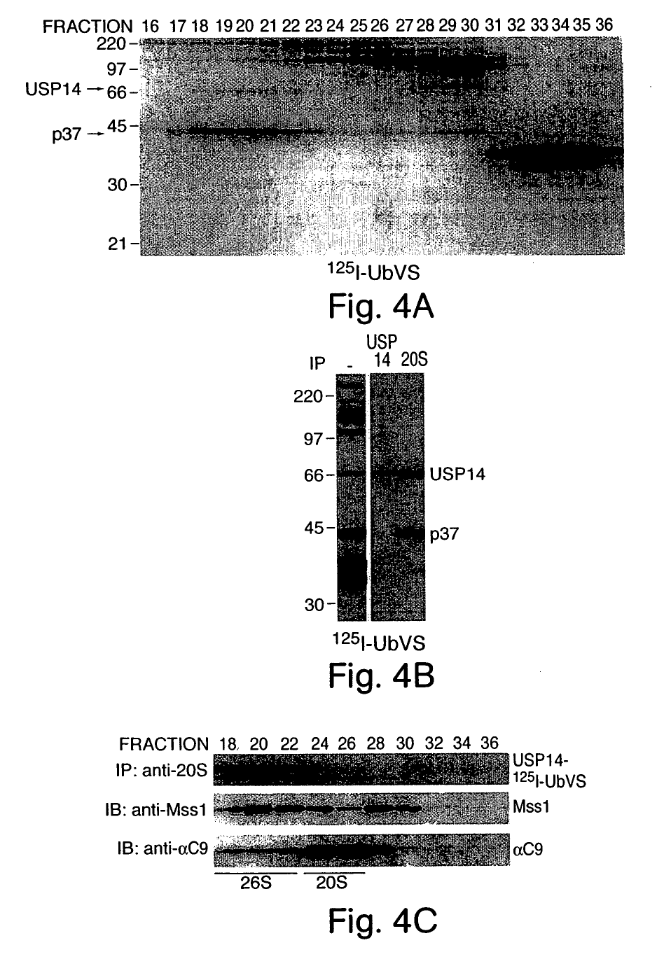

Labeling of a variety of mammalian cell extracts, prepared from tissues or cultured cells, revealed greater mammalian complexity of active DUB species (FIG. 3), reflecting a larger number of mammalian UBPs and UCHs (Chung et al., Biochem Biophys Res Commun, 266, 633-40, 1999). One labeled polypeptides herein was similar in size and affinity for Ubal as yeast Ubp6p (FIG. 2B and 3B). Immunological criteria confirm herein that this polypeptide corresponds to the mammalian homolog of Ubp6, USP14 (FIG. 4B). USP14 and Ubp6p have been studied in vitro, but their physiological function remains unclear. A deletion mutant of Ubp6 in yeast is viable; reported phenotypes include sensitivity to canavinine and stabilization of the Ub-Pro-β-galactosidase (Wyndham et al., Protein Sci, 8, 1268-75, 1999). In vitro studies of recombinant USP14 show that the monomeric protein has low affinity both for Ub and for non-hydrolyzable Ub dimers. Data herein with with HAUb probes showed relatively poor competition of UbVS labeling by Ubal. USP14 is apparently unable to disassemble poly-Ub protein conjugates in vitro (Yin et al., Biochemist, 39, 10001-10, 2000).

USP14 and its homologs possess a type II ubiquitin-like (Ubl) domain at the N-terminus (Jentsch and Pyrowolakis, Trends Cell Biol, 10, 335-42, 2000; Schauber et al., Nature, 391, 715-8,1998). Ubp6 lacking a Ubl does not complement Ubp6 deletion phenotypes, and Ubl is not necessary for in vitro processing of linear Ub fusions by Ubp6p (Wyndham et al., Protein Sci, 8, 1268-75, 1999). The Ubl may be necessary for targeting USP14/Ubp6 to its interacting partner or substrate, but not for intrinsic catalytic activity. Proteins containing a type II Ubl such as HPLICZ1, Rad 23 and BAG-1 require this domain for interaction with the 26S proteasome (Kleijnen et al., Mol Cell, 6, 409-19, 2000; Luders et al., J Biol Chem, 275,4613-4617, 2000; Schauber et al., Nature, 391, 715-8, 1998). The 19S subunit, S5a binds poly-ubiquitin chains, and a direct interaction between S5a and the Ubl of hHR23 has been reported, suggesting that S5a may also bind other Ubl containing proteins (Hiyama et al., J Biol Chem, 274, 28019-25, 1999). The binding of Rad23 to S5a does not lead to its degradation. Data show that the Ubl does not target USP14 for degradation by the proteasome, as 35S labeled USP14 is stable for long chase periods (624 hr).

Transient association of several DUBs with the 26S proteasome has been observed, including Doa4 in yeast and Ap-UCH in Aplysia (Hegde et al., Cell, 89, 115-26, 1997; Papa et al., Mol Biol Cell, 10,741-56, 1999). However, these enzymes are not detected in purified proteasome preparations. The only DUB considered to be a stable part of the 19S complex is a 37 kDa UCH found in pure proteasome from human and Drosophila sources, and has been localized to the hinge region between the lid and the base of the 19S (Holzl et al., J Cell Biol, 150, 119-130,2000; Lam et al., Nature, 385, 737-40, 1997). A Ubal-insensitive deubiquitinating activity associated with the 26S was reported in an earlier study, but its identity was never established (Eytan et al., J Biol Chem, 268, 4668-74, 1993). Low amounts of Ubp6p were detected by mass spectrometry performed on affinity purified yeast proteasomes (Verma et al., Mol Biol Cell, 11, 3425-39, 2000). However, USP14 was not detected in conventionally purified proteasomes, indicating that the USP14-proteasome association is less stable than that of p37, and that recovery of USP14 is highly dependent on the experimental method used. Proteins that interact with various of the reactive vectors herein and identified using genetically marked yeast strains and other considerations are listed in Tables 3 and 4.

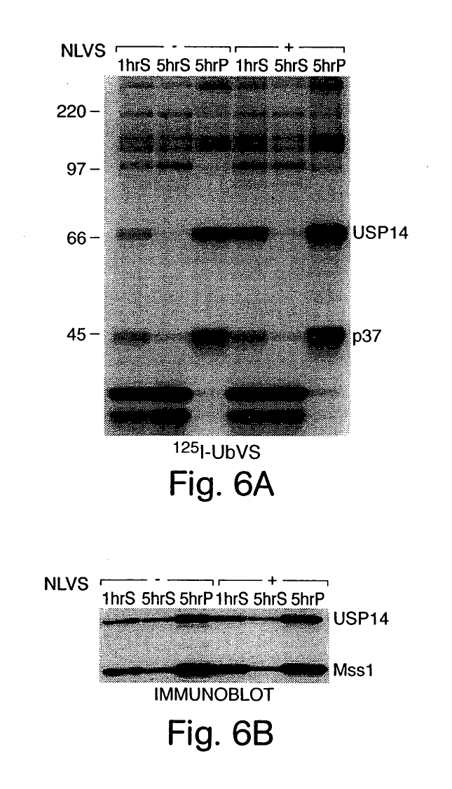

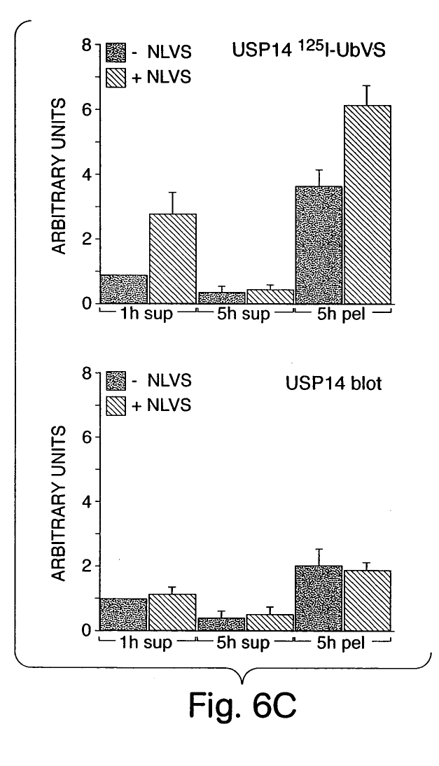

Labeling of p37 with [¹²⁵I]-UbVS in unaffected by the presence of proteasome inhibitors (FIG. 4A), whereas USP14 labeling is increased as much as 15-fold in a time dependant manner FIG. 4B). This difference in response to proteasome inhibition indicates that USP14 functions differently from p37, which has an activity thought to edit the poly-Ub chains on proteasome substrates by cleaving from the distal end of the chain (Lam et al., J Biol Chem, 272, 28438-46, 1997; Lam et al., Nature, 385, 737-40, 1997). Proteasome-associated USP14 is likely to interact with poly-Ub conjugated proteins as substrates. Preliminary observations indicate that only the proteasome bound form of USP14 can be labeled with [¹²⁵I]-UbVS (FIG. 6), suggesting that affinity of proteasome-bound USP14 for Ub may be different from that determined with the free recombinant protein (Yin et al., Biochemist, 39, 10001-10,2000). If so, USP14 is the first example of a DUB whose substrate specificity and activity are regulated by association with a binding partner.

The increase in USP14 labeling observed herein is not due to up regulation of de novo synthesis of USP14, as a similar increase is seen in cell free lysates treated with NLVS, and in whole cells in which translation is blocked by addition of puromycin (FIG. 6). The increase CD in labeling could be due to the stabilization of the proteasome complex in the presence of inhibitor, resulting in binding of additional USP14 protein.

Without limitation as to mechanism, a functional coupling between the activities of the proteasome and that of proteasome-bound USP14 is here proposed. Possible mechanisms to achieve such coupling, without limitation, include activation of USP14 by a conformational change propagated through the entire 26S complex upon proteasome inhibition. Evidence for the transmission of a conformational change upon engagement of HslU to the active site of HslV has recently been reported (Sousa et al., Cell, 103, 633-43, 2000), demonstrating the possibility of a long-range conformational change within a large protein complex. USP14 activity alternatively may be regulated by the level of poly-ubiquitinated substrates, which accumulate upon proteasome inhibition if Ub conjugation continues under our experimental conditions. The DUB specific affinity probe, UbVS, and others herein will be useful tools to further define the role of USP14 in proteasome mediated protein degradation.

Since the probe with vinyl sulfone was found not to target all possible DUBs, development of vectors having additional chemical groups is desirable. Introduction of epitope tags, or other tags in place of standard radioactive labeling would also facilitate visualizaiton and identification of enzymes, as well as providing other useful attributes to the probes, such as use of TAT protein from HIV to achieve cell permeability. Probes were designed to permit additional of inhibitors specific for individual enzymes, as well as for groups of proteins, so that specific diagnostic and therapeutic applications can be fulfilled.

Semisynthetic ubiquitin (Ub) and ubiquitin-like (Ubl) derivatives incorporating one of a diversity of chemical groups can be prepared by expressing the desired protein (Ub or Ubl) in E. coli using a commercially available intein-containing vector (obtained, for example, from New England Biolabs, Beverly, Mass.; Single-column purification of free recombinant proteins using a self-cleavable tag derived from a protein splicing element. S. Chong et al. Gene 192, 1997, 271-281). Expressed protein can contain, in order from the N- to the C-terminal of the construct: an N-terminal epitope-tag, a desired affinity-creating binding peptide such as the Ub/Ubl sequence lacking the C-terminal amino acid, an intein and a chitin-binding domain. After purification over chitin-beads, the Ub/Ubl polypeptide is cleaved from the intein using MESNA (mercapto ethane sulfonic acid sodium salt).

Thus obtained, Ub/Ubl proteins are modified by organic chemistry techniques to incorporate a C-terminal chemical reactive group (electron withdrawing group (EWG) acting as a “warhead”). The resulting semi-synthetic product is capable of interacting with Ub/Ubl specific enzymes in a reversible fashion (for example, the product is a peptide having aldehydes/boronates) or is capable of interacting with Ub/Ubl specific enzymes in an irreversible fashion (the product is a peptide-Michael acceptor, alkylating agent). The modified enzymes (in the case of irreversible inhibitors) can be visualized by SDS-PAGE followed by an immunoblot with antibodies directed against the N-terminal epitope tag, for example, hemagglutinin (HA). Cell lysates treated with the Ub or Ubl-derived probes can be generated from a wide variety of types of cells and cell tissues. The probes and methods herein can be used to determine the activities of various proteins in crude extracts, for diagnostic purposes. Further, probes can be used to localize proteins, for example, during cell fractionation to separate cell membrane, cytoplasmic, nuclei, nuclear membrane, and mitochondrial fractions, for further localization of specific enzymes.

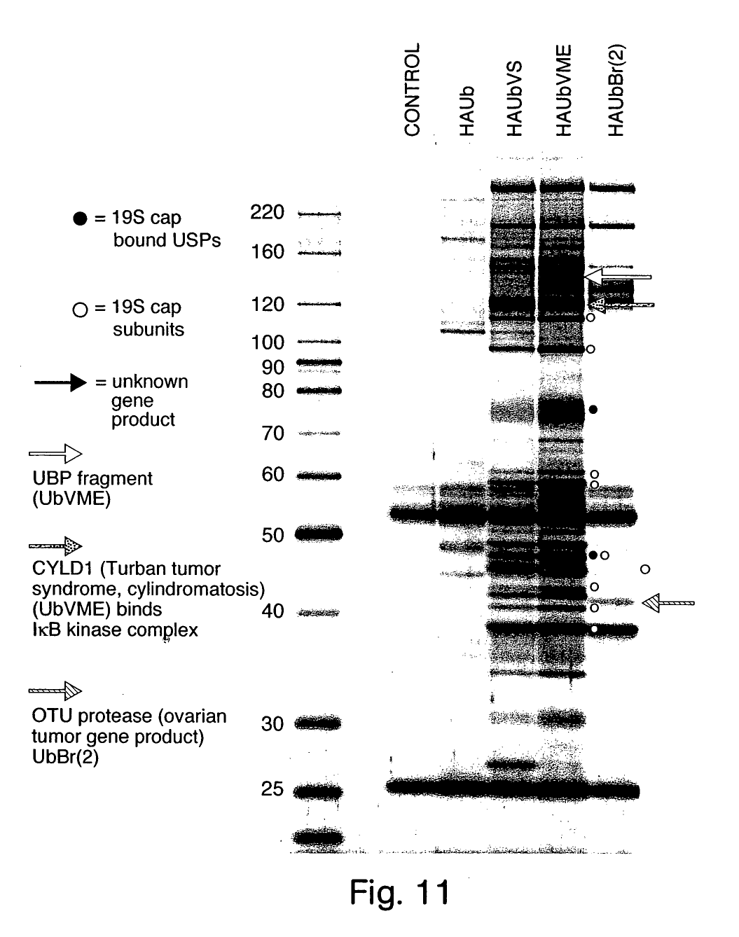

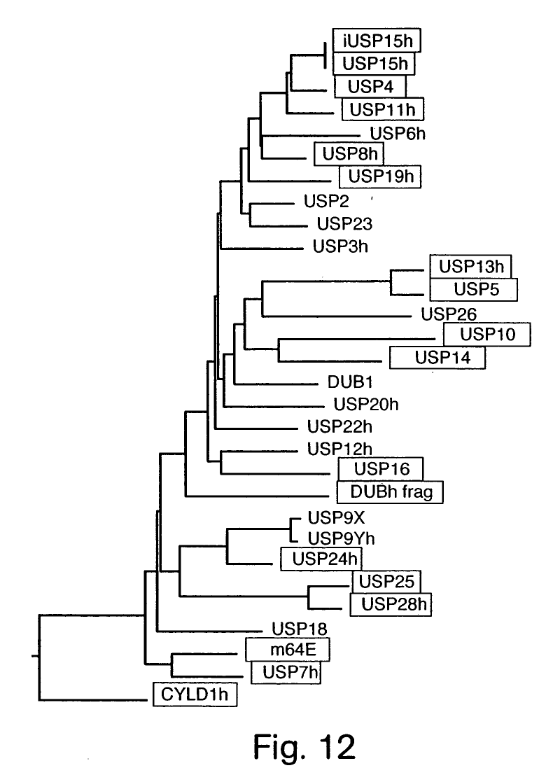

Enzymes and proteins modified by interaction with the probes herein can be further immunoprecipiated by an antibody specific for the N-terminal epitope tage, and can be visualized by SDS-PAGE followed by staining, for example, silver stain (FIG. 11), and can be further identified by tandem mass spectrometry (FIG. 12 and Tables 3 and 4).

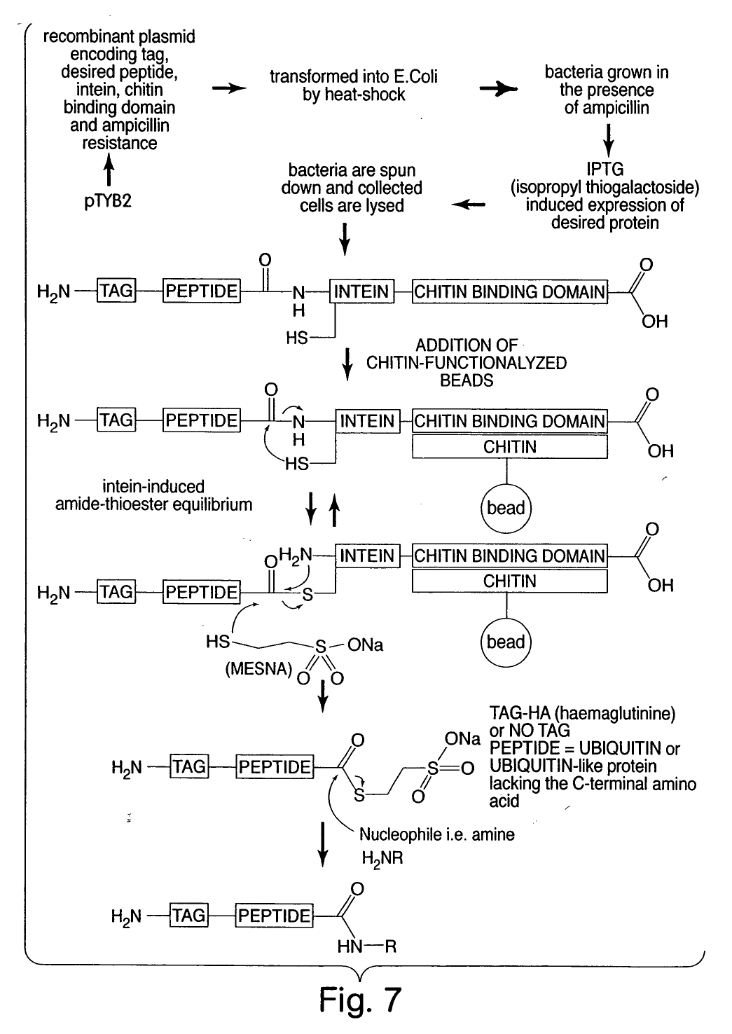

It is here shown that functionalyzed Ub proteins efficiently label enzymes involved in Ub-conjugation and deconjugation. It is here also shown that this method allows for the large-scale preparation of full-length C-terminally unmodified Ub with or without N-terminal epitope tag. See FIG. 7.

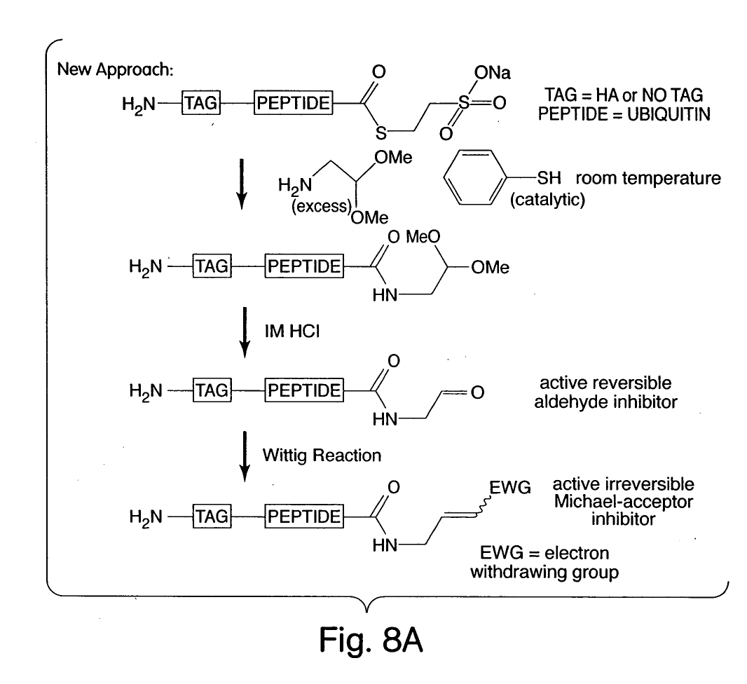

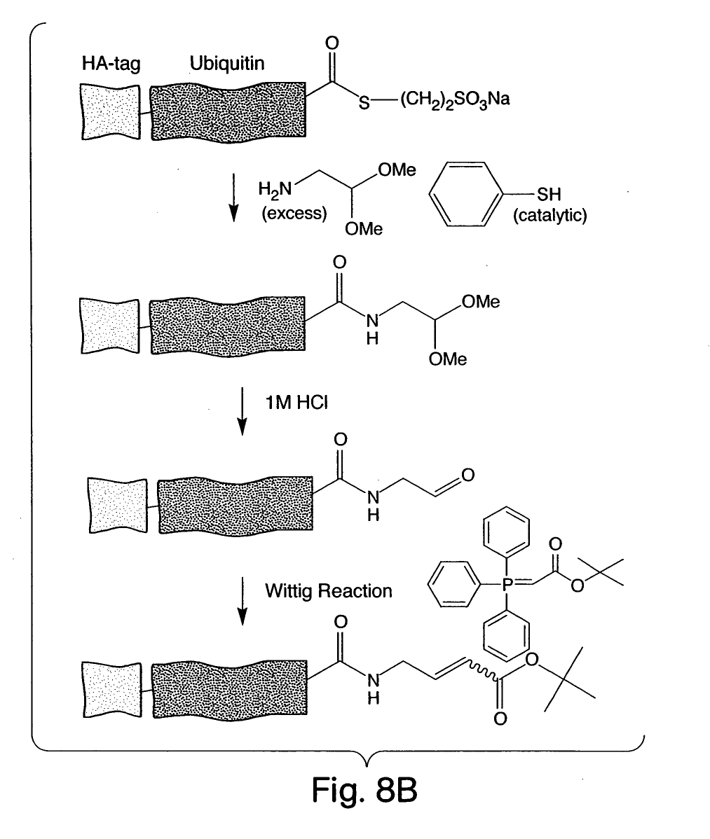

Warheads specific to a proteomic subset, for example the ubiquitin-related proteome, can be introduced either by direct nucleophilic substitution of intein-derived sequences, for example, Ub thioesters, or by the following steps: adding 1-amino-2,2dimethoxyalkane; performing acid catalyzed hydrolysis of the resulting acetal; and isolating peptide aldehydes (reversible inhibitors of a variety of ubiquitin-related enzymes). See FIG. 8.

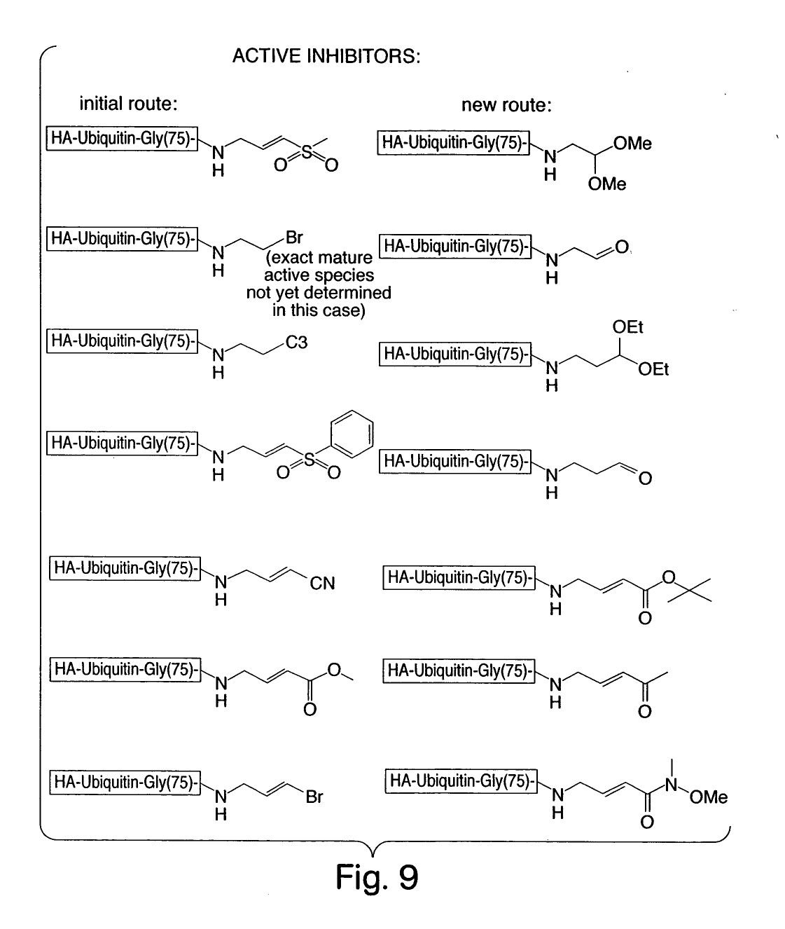

Further, use of Wittig reaction with stabilized ylides of the thus obtained aldehydes in aqueous solution directly leads to peptide-Michael acceptors (which are irreversible inhibitors). See also the attached synthetic schemes. See FIG. 2. Examples of inhibitors obtained by these methods are shown in FIG. 9.

The invention now having been fully described, specific emodiments are shown in the Examples below, which are not to be construed as further limiting. A portion of this work was published Sep. 17, 2001, as “A novel active site-directed probe specific for deubiquitylating enzymes reveals proteasome association of USP14.” A. Borodovsky et al. Embo J., 2001, 20:5187-5196, and “Chemistry-based functional proteomics reveals novel members of the deubiquitinating enzyme family”, A. Borodovsky et al. Chem. Biol, 2002, 9:1-20, the contents of both of which are incorporated herein by reference in their entirety.

### EXAMPLES

The following materials and methods were used throughout the Examples herein. Materials. HPLC-grade organic solvents N,-dimethyl formamide (DMF),and dichloromethane (American Bioanalytical), n-hexane and ethyl acetate (Fisher) were used as received Tetrahydrofuran was obtained from Acros and distilled over LiAlH₄ under a m nitrogen atmosphere prior to use. N,N-dimethylaminopyridine (DMAP) was purchased from Lancaster. Thiophenol, 2-chloroethylamine hydrochloride and 2-bromoethylamine hydrobromide were purchased from Acros. 3-bromopropylamine hydrobromide, methyltriphenylphosphoranylidene acetate and triphenylphosphoranylidene acetonitrile were purchased from Aldrich. Methanesulfonylmethyl-phosphonic acid diethyl ester and benzenesulfonylmethyl-phosphonic acid diethyl ester were synthesized according to literature procedures (Dragovich et al., 1998, J. Med. Chem. 41:2806-2818; Liu and Hanzlik, 1992, J. Med. Chem. 35:1067-1075). Slide-a-lyzer™ dialysis membranes were obtained from Pierce. NMR spectra were recorded on a Varian 200 MHz spectrometer, mass spectra were recorded on an electrospray LCZ LC-MS instrument (LC HP1100 Hewlett Packert, MS Micromass, UK) equipped with a Waters DeltaPak C4 (3.9×50 mm) column.

Synthesis of Thiol-Reactive Groups for Chemical Ligtion to HAUb₇₅MESNa.

N-tert-butyloxycarbonyl (Boc) protected glycinal was obtained by sodium periodate-mediated oxidative diol cleavage of N-Boc-1-amino-2,3-propanediol. Thus obtained N-Boc-glycinal was reacted with either Wittig ylides (methyl triphenyl phosporanylidene acetate for VME, triphenylphosporanylidene acetonitrile for VCN) at room-temperature or with Horner-Wadsworth-Emmons reagents (Methanesulfonylmethyl-phosphonic acid diethyl ester, sodium hydride for HAUbVS, benzenesulfonylmethyl-phosphonic acid diethyl ester, sodium hydride for HAUbVSPh) in THF at 0° C. in all cases resulting in high yields ˜80% of the N-Boc protected electrophilic traps as Z/E mixtures. The E-isomer was in all cases the major isomer and could be purified by column chromatography on silica gel. Deprotection of the Boc-groups was performed by adding dry toluenesulfonic acid (three equivalents, no stirring) in either diethylether or methyl-tert-butyl ether upon which the deprotected amines crystallize as para-toluene sulfonic acid salts.

Plasmid Construction.

pTYB-HAUb plasmid was constructed by cloning the sequence of human Ub (lacking Gly76) into the pTYB2 vector (New England Biolabs) to generate an in-frame fusion with the intein and chitin-binding domain. The HA tag was introduced by inserting an oligonucleotide cassette into the NdeI site at the 5′ end of the Ub sequence.

Synthesis of HAUb₇₅-MESNa.

A single colony of pTYB-HAUb was grown in 1 L of LB medium containing 100 μg/ml ampicillin at 37° C. and expression was induced for 2 hours at 30° C. by the addition of 100 mg isopropyl-β-thio-galactopyranoside. Cells were spun down (4000 rpm) and resuspended in 50 ml 50 mM HEPES pH 6.5, 100 mM NaOAc, 50 μM PMSF and lysed in a french press. (1500 psi). A clarified cell extract was obtained by centrifugation (12000 rpm). The cell extract was loaded onto a 15 ml chitin bead (New England Biolabs) column at a flow rate of 0.5 ml/min. The column was washed with 60 mL of lysis buffer followed by 25 mL of lysis buffer containing 50 mM β-mercaptoethanesulfonic acid sodium salt (MESNa) and incubated overnight at 37° C. for the induction of on-column cleavage. 25 mL of lysis buffer was used to elute the HAUb₇₅-MESNa thioester, the eluent was concentrated to 1 mL to give approximately 2.5 mg of protein. The N-terminal methionine of the HA-tag was frequently partially processed off, giving a mixture of two proteins that behave identically in labeling experiments.

Synthesis and Purification of HAUb-Derived Active Site Thiol-Reactive Probes.

HAUbCl. HAUbBr2. HAUbBr3: To a solution of HAUb₇₅-MESNa (1-2 mg/mL) in column buffer (500 μL), was added subsequently: 0.2 mmol of the desired haloalkylamine haloacid salt and 100 μl of 2.0 M aqueous NaOH and the mixture was immediately vortexed. After 20 minutes at room temperature. 100 μl of 2.0 M aqueous HCl was added and the solution was dialyzed against 50 mM NaOAc, pH 4.5 in a 3.5 mL Pierce Slide-a-lyzer cassette (3500 MWCO) for two hours. The resulting product (>90% conversion estimated from LC-MS) was divided into aliquots and stored at −80° C. (no significant deterioration is observed for several months of storage). HAUbVME, HAUbVS, HAUbVCN: To a solution of HAUb₇₅-MESNa (1-2 mg/ml, 500 pi), was added subsequently: 0.125 mmol of the desired Michael acceptor as para-toluene sulfonic acid salt followed by 75 μL of 2M N-hydroxy succinimide and 125 μL 2 M NaOH. The mixture was incubated at 37° C. for 2 hours and reaction progress was monitored by LC-MS to give the desired products with 50-60% conversion accompanied by hydrolysis. The reaction mixture was neutralized by the addition of 125 μL of 2 M HCl and dialyzed as described above. HAUbVsPh: To a solution of HAUb₇₅-MESNa (1-2 mg/ml, 500 μl), was added subsequently: a solution of glycine vinyl phenyl sulfone tosic acid salt (0.2 mmol, 46 mg) in 250 μl of DMF followed by 50 μl of 1 M DMAP in DMF and 100 μl of 2 M aqueous NaOH. Reaction progress was monitored by LC-MS and after 45 minutes 100 μl of 2 M aqueous HCl and 1 ml of 50 mM NaOAc pH 4.5 was added and the mixture was dialyzed overnight at 4° C. against the same solvent. The product was filtered, and W concentrated to approximately 500 μl. HAUb: To a solution of HAUb₇₅-MESNa (1-2 mg/ml, 500 μl ), was added subsequently 200 μl of 2.3 M glycine pH 8.3 in water containing 2 μl thiophenol. After 3 hours the mixture was filtered and dialyzed as described above. HAUb, HAUbVS, HAUbVSPh, HAUbVME, HAUbVCN, HAUbBr2, HAUbBr3 were purified to 95% purity using a Pharmacia SMART system MonoS 1.6/5 column with a linear gradient from 0 to 30% B; 50 mM NaOAc pH 4.5 (buffer A), 50 mM NaOAc pH 4.5, IM NaCl (buffer B), HAUbVSPh gave a different elution profile due to the hydrophobicity of its C-terminus. All synthetically modified HA-tagged ubiquitin derivatives were purified before they were used in experiments with the exception of HAUbBr2, which appeared to be less stable. This compound was used directly after dialysis, since the synthesis yield was >90%. All Ub-derived probes were analyzed by LC-MS (ESI). A C4 reverse phase HPLC column with a 0-80% gradient over 20 min using a 0.1% formic acidlacetonitrile buffer system was used at a flow rate of 0.4 ml/min. Multi-charges species observed for each HAUb-derived probe are given (species listed contain the N-terminal methionine residue): Ub-MESNa: found (calculated); [M+9H])⁹⁺ 960 (960); [M+8H]⁸⁺ 1079 (1080); [M+7H]⁷⁺ 1233 (1234); [M+6H]⁶⁺ 1439 (1440). HAUb-MESNa: found (calculated) [M+11H]¹¹⁺ 936 (936); [M+10H])¹⁰⁺ 1029 (1029); [M+9H]⁹⁺ 1143 (1144); [M+8H]⁸⁺ 1286 (1287); [M+7H]⁷⁺ 1469 (1470). HAUb: Found (calculated) [M+11H¹¹⁺ 929 (928); [M+10H])¹⁰⁺ 1022 (1021); [M+9H]⁹⁺ 1135 (1134); [M+8H]⁸⁺ 1277 (1276); [M+7H]⁷⁺ 1459 (1458). HAUbVS: found (calculated); [M+11H]¹¹⁺ 935 (935); [M+10H]¹⁰⁺ 1027 (1029); [M+9H]⁹⁺ 1142 (1143); [M+8H]⁸⁺ 1286 (1286); [M+7H]⁷⁺ 1468 (1469). HAUbVME: found (calculated) [+11H]¹¹⁺ 933 (933); [M+9H]¹⁰⁺ 1027 (1027); [M+9H]⁹⁺ 1140 (1141); [M+8H]⁸⁺ 1283 (1283); [M+7H]⁷⁺ 1466 (1466). HAUbVCN: found (calculated): [M+11H]¹¹⁺ 932 (930); [M+10H]¹⁰⁺ 1025 (1023); [M+9H]⁹⁺ 1137 (1137); [M+8H]⁸⁺ 1280 (1279). HAUbCl: Found (calculated): [M+11H]¹¹⁺ 930 (930); [M+10H]¹⁰⁺ 1023 (1023); [M+9H]⁹⁺ 1337 (1137); [M+8H]⁸⁺ 1279 (1279). HAUbBr2: found (calculated); [M+11H]¹¹⁺ 934 (934); [M+10H]¹⁰⁺ 1027 (1027); [M+9H]⁹⁺ 1141 (1142); [M+8H]⁸⁺ 1284 (1284); [M+7H]⁷⁺ 1467 (1468). HAUbBr3: found (calculated): [M+11H]¹¹⁺ 935 (936); [M+10H]¹⁰⁺ 1028 (1029); [M+9H]⁹⁺ 1286 (1286); [M+8H]⁸⁺ 1469 (1469). HAUbVsPb: found (Calculated): [M+11H]¹¹⁺ 940 (941); [M+10H]¹⁰⁺ 1034.(1035); [M+9H]⁹⁺ 1149 (1150); [M+8H]⁸⁺ 1292 (1293); [M+7H]⁷⁺ 1477 (1478).

Preparation of EL-4 Cell Extracts and Labeling with HAUb Derivatives.

EL4 cells (cultured in RPMI-HEPES supplemented with 10% FCS, 1% glutamine and 1% penicilline/streptomycine) were harvested and washed 3× with PBS. Cell pellets were lysed with glass beads in buffer HR (50 mM Tris pH 7.4, 5 mM MgCl₂, 250 mM sucrose, 1 mM DTT, 2 mM ATP). Nuclei were removed by centrifugation and 20 μg protein extract was used for labeling with HAUb derivatives. Indicated concentrations of HAUb derivatives were incubated with cell extracts for 1 hr at 37° C. Samples were resolved by reducing 8% SDS-PAGE, blotted onto PVDF membranes, blocked with 5% milk 0.1% Tween in PBS and incubated with anti-HA 12CA5 monoclonal antibody. Detection was by chemiluminesence, using goat-anti-mouse-HRP as secondary antibody.

Anti-HA Immunoprecipitation for Tandem Mass Spectrometry Analysis.

EL4 cell lysates were prepared as above, except 0.5×10⁹-2×10⁹ cells were used and 50 μM PMSF was included in the lysis buffer. Lysates (at around 5 mg/ml) were incubated with the desired HAUb-derived probe (5 mg lysate and 6.6 μg of the probe were used for silver stains, 14-20 mg lysate and 20 μg of probe were used for Coomassie stains) for 2 hrs at 37° C. SDS was added to “denatured” samples to the final concentration of 0.4% and then diluted to less then 0.1% with NET buffer (50 mM Tris pH 7.5,5 mM EDTA, 150 mM NaCl, 0.5% NP40) prior to the addition of anti-HA agarose. Anti-HA agarose (Sigma) was incubated with the samples overnight at 4° C., the immunoprecipitations were washed extensively with NET buffer and the bound proteins were eluted with 50 mM Glycine pH 2.5 at 4° C. for 30 min. All samples were evaporated to dryness and redisolved in 50 μl of 1× SDS-PAGE sample buffer. pH was adjusted with 1M Tris pH 8 if needed. Samples were resolved by 8% reducing SDS-PAGE and stained with silver or Commassie stain using standard conditions.

Protein Identification by Tandem Mass Spectrometry.

Individual polypeptides were excised from gels, destained and subjected to trypsinolysis by standard biochemical procedures. The samples were separated using a nanoflow liquid chromatrography system (Waters Cap LC, Medford, Mass.) equipped with a picofrit column (75micron ID, 10 cm, NewObjective, Woburn, Mass.) at a flow rate of approximately 150 nl/min using a nanotee (Waters, Medford, Mass.) 16/1 split (initial flow rate 5.51 μl/min). The LC system was directly coupled to a tandem mass spectrometer (Q-TOF micro, Micromass, Manchester, UK). Analysis was performed in survey scan mode and parent ions m with intensities greater than 6 were sequenced in MS/MS mode using MassLynx 3.5 Software I (Micromnass, UK). MS/MS data were processed and subjected to database searches using ProteinLynx Global Server 1.1 Software (Micromass, UK) against Swissprot, TREMBL/New (http://www.expasy.ch), or using Mascot (Matrixscience) against the NCBI non-redundant (nr) or mouse EST databases.

Cell lines and antibodies. EL-4 (a mouse thymoma line from thymic epithelium) and NIH3T3 mouse cell lines were maintained under standard cell culture conditions (Bogyo et al., Chem Biol, 5, 307-20, 1998). The rabbit anti-human HAUSP peptide serum r201 (Everett et al., Embo J, 16, 566-71, 1997), and rabbit anti-mouse 20S proteasome and anti-αC9 anti-sera (Nandi et al., Embo J, 16, 5363-75, 1997) were obtained. Rabbit anti-serum against Mss1 was purchased from Affinity Research Products Ltd (Exeter, UK). Anti-serum against mouse USP14 (HM433 and HM434) was raised in NZW rabbits immunized with keyhole limpet hemagglutinin (KLH) coupled to four synthetic peptides each having the position of the amino acid residues indicated by subscripts of the USP14. sequence Y₃SVTVKWGKEKFEGVELNT₂₁C (SEQ ID NO: 1); CK₂₃₉SLIDQYFGVEFETTMK₂₅₆ (SEQ ID NO: 2); CK₂₈₉LRLQEEITKQSPTLQRNAL₃₀₈ (SEQ ID NO: 3); and C₃₅₈TPELQEKMVSFRSKFKDLED₃₇₈ (SEQ ID NO: 4), each peptide synthesized on an Advanced ChemTech 440 MOS synthesizer (Louisville, Ky., USA).

Inhibitors. The proteasome inhibitors NLYS and ZL₃VS were synthesized as described (Bogyo, Thesis, Massachusetts Institute of Technology, 1997), and epoxomicin and ubiquitin aldehyde were purchased from Affinity Research Products Ltd (Exeter, UK).

Yeast straits, media and methods. Media were prepared as described (Sherman, Methods in Enzymology., 194, 3-21, 1991). All yeast cultures were grown in rich media (YPD) at 30° C. A wild-type strain MHY501 (Mata, his3-Δ200, leu2-3,112, ura-52, lys2-801, trpI-1) was used. Strains with deletions in UBP genes were otherwise genetically identical to MHY501 (MHY526, Δubp1::URA3; MHY648, Δubp2::TRP1; MHY821, Δubp6::HIS3; MHY887, Δubp12::HIS3; MHY989, Δubp15::HIS3; MHY525, Δuh1::LEU2).

To prepare yeast lysates, 8 OD of exponentially growing yeast cells were harvested. Cells were resuspended in PBS containing 2 mM ATP, 1.5 mM DTT, 20 mM PMSF, 1 μM TPCK, 1 μM Leupeptin, 1 μM Pepstatin, and were lysed by vortexing with glass beads. Lysates were centrifuged to remove cell debris and nuclei. An amount of 50-100 μg of lysate was used for labeling with [¹²⁵I]-UbVS.

Purification of UCH-L3. The E. coli expression vector for UCH-L3 and methods for overexpression in E. coli and purification are as described (Larsen et al., Biochemist, 35, 673544, 1996), with use of Q Sepharose and Sephadex 75 FPLC columns. An amount of 66 ng of UCH-L3 was used for labeling with [¹²⁵I]-UbVS.

Synthesis of Ubiquitin vinyl sulfone (UbVS). Ubiquitin₇₅ ethyl ester (Ub₇₅OEt) was synthesized and purified by gel filtration and cation exchange chromatography (Wilkinson et al., Biochemistry, 25, 6644-9, 1986; Wilkinson et al., Biochemist, 29, 7373-80, 1990), with addition of a final purification step using a Pharmacia MonoS 1.6/5. Ub₇₅OEt was converted to Ub₇₅ hydrazine as described and was used without further purification (Wilkinson et al., Biochemist, 29, 7373-80, 1990). Trans-Boc-Gly-VS was synthesized (Bogyo et al., Chem Biol, 5, 307-20, 1998) and was deprotected prior to use by treatment with 50% trifluoroacetic acid in methylene chloride or treatment with p-toluene sulfonic acid. Ub75 hydrazine was converted to Ub75 azide by treatment with 0.5 M nitrous acid at −5° C. for 1 min, which was immediately coupled with NH₂Gly-VS in the presence of triethylamine (Wilkinson et al., Biochemist, 29, 7373-80, 1990). After a 5 min incubation at −5° C., the reaction was dialyzed against 50 mM sodium acetate pH 4.5 and purified on the Pharmacia SMART system MonoS 1.6/5 column to 95% purity. UbVS was identified by liquid chromatography/mass spectroscopy, using a LCZ electrospray mass spectrometer instrument (Micromass, UK) coupled with an HP1100 HPLC system (Hewlett Packard, USA). A C4 reverse phase HPLC column with a 0-80% gradient over 20 min and a 0.1% formic acid/acetonitrile buffer system was used.

Preparation of mammalian cell extracts and labelings with ¹²⁵I]-UbVS. An amount of 5×10⁸ EL4 cells was harvested and washed three times with PBS. Cell pellets were lysed with glass beads in buffer HR (50 mM Tris pH 7.4, 5 mM MgCl₂, 250 mM sucrose, 1 mM DTT, 2 mM ATP). Nuclei were removed by centrifugation, and an amount of 20 μg of lysate was used for labeling with [¹²⁵I]-UbVS. An amount of 40 μg UbVS was iodinated as described for Ub (Ciechanover et al., Proc Natl Acad Sci USA, 77,1365-1368,1980), using Iodo-gen as a catalyst, and 1 mg/ml hen egg lysozyme was added as carrier protein after quenching the reaction. An amount of0.5×10⁶-1×10⁶ cpm of ¹²⁵I-UbVS was incubated with cell extracts for 1 hr at 37° C. Samples were resolved by electrophoresis using reducing SDS-PAGE, and were analyzed by autoradiography.

Immunoprecipitation. Anti-proteasome immunoprecipitations (IP) were carried out using 80 μg of EL-4 lysates prepared as above, previously labeled with 2×10⁶ cpm of ¹²⁵I-UbVS or ¹²⁵I-NLVS. IP conditions described by Luders and co-workers were used (Luders et al., J Biol Chem, 275, 4613-4617, 2000). Briefly, samples were resuspended in proteasome IP buffer (25 mM Tris pH 7.5, 100 mM KCl, 0.5% Tween 20, 2 mM MgCl₂, 1 mM ATP, 1 mM PMSF, 2 μg/ml aprotinin, 0.5 μg/ml leupeptin), and protease inhibitors were further omitted after the pre-clearing step. Samples were precleared two times with normal rabbit serum and were immunoprecipitated with 3 μl of anti-mouse 20S proteasome serum, and immune complexes were recovered with fixed Staphylococcus aureus (Staph A). Pellets were washed 3× with 1 ml of proteasome IP buffer before addition of SDS-PAGE sample buffer, followed by analysis by SDS-PAGE and autoradiography. Immunoprecipitations with anti-USP14 antibodies were carried out on lysates denatured with 1% SDS in PBS. SDS concentration was decreased to less than 0.01% with NP40 lysis buffer (10 mM Tris pH 7.8, 0.5% NP40, 150 mM NaCl, 5 mM MgCl₂) for subsequent pre-clear and immunoprecipitation. StaphA immunoprecipitates were washed 3× with NET buffer (0.5% NP40, 50 mM Tris pH 7.4, 150 mM NaCl, 5 mM EDTA).

Immunoblotting. Immunoblots were carried out according to published protocols (Bonifacino, Current Protocols in Cell Biology. John Wiley & Sons, Vol. 1, pp. 6.2.1-6.2.1 6, 2000). Briefly, samples were resolved by SDS-PAGE and blotted onto PVDF membranes, membranes were blocked with 5% milk, 0.1% Tween in PBS, and were incubated with primary antibody at 1:1000 dilution unless otherwise indicated. Detection was by chemiluminesence, using goat-anti-rabbit-HRP as secondary antibody. For the anti-USP7 immunoblot, 20 μg of EL4 cell lysate was pre-incubated for 1 hr with or without 2 μM UbVS, and the reaction was resolved by SDS-PAGE and immunoblotted for USP7 (HAUSP) with r201 anti-serum as described (Everett et al., Embo J, 16, 566-77, 1997).

Subcellular fractionations. Proteasome fractions were generated as described (Wang et al., Proc Natl Acad Sci U S A, 97, 9990-5, 2000). Briefly, EL4 cells were lysed in proteasome homogenization buffer, lysates were centrifuged for 1 hr at 100,000 g to pellet membranes, and the supernatants were centrifuged for an additional 5 hrs at 100,000 g to produce a pellet having the proteasome enriched fraction.

Superose 6 column. EL4 lysates were centrifuged for 1 hr at 100,00×g and were fractionated in proteasome homogenization buffer on a preparative Pharmacia Superose 6 column using the AKTA FPLC system (Pharmacia, Sweden).

### Example 1

### Synthesis and Characterization of UbVS

The strategy for the synthesis of UbVS is outlined in FIG. 1A. Ubiquitin, compound 1, was digested with trypsin in presence of 2.5M glycine ethyl ester to obtain Ub₇₅-ethyl ester, compound 2, in approximately 40% yield (Wilkinson et al., Biochemhistry, 25, 6644-9, 1986). Trypsin was inactivated by addition of soybean trypsin inhibitor and PMSF. Ub₇₅-ethyl ester was purified by Sephadex G-50 gel filtration and CM Sepharose cation-exchange chromatography. Treatment of compound 2 with hydrazine converted the ethylester moiety into a hydrazine, yielding compound 3, and subsequent exposure of m compound 3 to dilute nitrous acid yielded Ub₇₅-azide, compound 4 (Wilkinson et al., Biochemist, 29, 7373-80, 1990). Both reactions were near quantitative, and little residual Ub-ethylester and hydrazine were detected. Compound 6, UbVS, was obtained by coupling compound 4 with an excess of NH₂-glycyl-vinyl sulfone, compound 5 (Bogyo et al., Chem Biol, 5, 307-20, 1998). Purification by cation-exchange chromatography allowed separation of UbVS from the remaining precursors and side products generated by the Michael reaction of additional glycine vinyl sulfone molecules with UbVS.

The molecular weight of the isolated UbVS was in agreement with the predicted mass, as assessed by liquid chromatography/electrospray mass spectrometry (LC/ES-MS; FIG. 1B). Overall yield was 3%.

### Example 2

### UbVS Specifically Labels the Ubiquitin C-Terminal Hydrolase UCH-L3

The UCH-L3 enzyme was produced in recombinant form in E. coli and purified to apparent homogeneity (Larsen et al., Biochemist, 35,6735-44, 1996). UbVS was radioiodinated with Na[¹²⁵I] and was added in substoichiometric amounts to UCH-L3. The appearance of an additional polypeptide of a molecular mass that was predicted for a covalent UCH-L3-UbVS adduct was observed on silver stained gels (FIG. 1C, left panel).

The reaction was blocked completely by inclusion of the alkylating agent N-ethylmaleimide, as predicted for the presence of an active site cysteine in UCH-L3 that was involved in the reaction. Autoradiography of the same samples confirmed that the UCH-L3-[¹²⁵I]-UbVS adduct was the single predominant radiolabeled species (FIG. 1C, right panel).

The data show that UbVS efficiently labels the Ub C-terminal hydrolase UCH-L3 through a Michael reaction between the active site thiol of UCH-L3 and the vinyl sulfone moiety. The resulting thioether is stable under the conditions routinely used for reducing SDS-PAGE.

### Example 3

### Labeling of Cell Extracts from Yeast Strains having Deletions of Known DUB Genes

The yeast genome specifies 16 UBPs and a UCH, Yuh1 (Chung et al, Biochem Biophys Res Commun, 266, 633-40, 1999). Yeast strains deficient in each of the genes encoding these enzymes were used to prepare cytosolic extracts, which were then exposed to [¹²⁵I-UbVS.

At least 5 clearly labeled polypeptides were observed, and were assigned to Yuh1p and UBPs 1, 2, 6 and 15 based on analysis of respective mutant strains (FIG. 2A; Table 2). On longer exposures, labeling of Ubp12p was also detected. These results provide genetic corroboration of the specificity of labeling by UbVS of the DUBs (UBPs and UCHs).

Specificity of labeling was further demonstrated by inclusion of ubiquitin aldehyde (Ubal), a known inhibitor of DUBs (Hershko and Rose, Proc Natl Acad Sci USA, 84, 1829-33, 1987). Labeling of DUBs by [¹²⁵I]-UbVS was found to be competed by pre-treatment with Ubal, the competition occuring in a concentration dependent manner (FIG. 2B). Ubp6p was less susceptible to competition than the other UBPs. The data show that UbVS labels only 5 prominent polypeptides in wild type yeast, even though 17 known DUBs are encoded by the yeast genome (Table 1).

### Example 4

### Labeling of DUBs in Mammalian Cell Extracts

Extracts were prepared from different mouse tissues and labeled with [¹²⁵I-UbVS (FIG. 3A).

Multiple polypeptides with significant differences in labeling patterns were observed for different tissues, the most striking of which was seen for brain (FIG. 3A lane 2). The intensely labeled polypeptide detected at a molecular weight (MW) of 30 kDa corresponds to the UCH-LI enzyme, known to be abundantly expressed in brain (Wilkinson et al., Science, 246,670-3, 1989).

At least 12 bands were observed upon longer exposures of autoradiograms, a substantially larger number of labeled species. As seen with yeast, labeling was abolished by inclusion of Ubal (FIG. 3B), establishing the specificity of labeling to DUBs. While confirming the known complexity of the DUB family, this observation demonstrates that active DUBs can be detected conveniently in a crude extract by incubation with [¹²⁵I]-UbVS, and can be analyzed by SDS-PAGE and autoradiography.

To demonstrate modification of a known UBP by UbVS, EL-4 cell lysates were incubated with UbVS, followed by immunoblotting with anti-serum specific for USP7 (HAUSP; Everett et al., Embo J, 16, 566-77, 1997). A shift in one of the two immunoreactive species, consistent with the formation of an USP7-UbVS conjugate, was observed (FIG. 3C). The upper polypeptide cross-reacting with anti-USP7 serum was previously observed (Everett et al., Embo J, 16, 566-77, 1997) and was not here reactive with UbVS.

These data demonstrate that UbVS labels both mammalian UCHs and UBPs, as shown here for USP7.

### Example 5

### Association of DUBs with Higher Molecular Weight Complexes

Several reports have described the association of DUBs with the proteasome (Hegde et al., Cell, 89, 115-26, 1997; Papa et al., Mol Biol Cell, 10, 741-56, 1999; Verma et al., Mol Biol Cell, 11, 3425-39, 2000; Voges et al., Annu. Rev. Biochem, 68, 1999). A DUB of 37 kDa (referred to here as p37) is thought to be a subunit of the 19S cap (Holzl et al., J Cell Biol, 150, 119-130, 2000; Lam et al., Nature, 385, 737-40, 1997). Since [¹²⁵I]-UbVS labeling of cell extracts herein allows monitoring of the activity of multiple DUBs at the same time, it is possible using the methods herein to directly examine the association of DUBs with higher molecular weight complexes, such as the 26S proteasome.

Labeling of EL-4 cell extract fractions obtained by gel filtration on a Superose 6 column with [¹²⁵I]-UbVS shows that most DUBs elute at positions consistent with their observed individual molecular mass, as determined by SDS-PAGE (FIG. 4A). However, two polypeptides (observed here as having MW of 45 and 66 kDa, respectively, each of which includes 8.5 kDa for UbVS) appear to be part of a larger complex (FIG. 4A, fractions 17-23). The “45 kDa” polypeptide (subtracting 8.5 kDa for UbVS) is identical in mass to the ubiquitin isonitrile-modified p37 DUB present in the 19S cap (Lam et al., Nature, 385, 737-40, 1997). This polypeptide is identified herein as the mammalian homologue of a UCH found in the 19S caps of Drosophila and S. pombe proteasomes (Holzl et al., J Cell Biol, 150, 119-130, 2000; Li et al., Biochem Biophys Res Commun, 272,270-5, 2000). The identity of the “66 kDa” (58 kDa) DUB was further characterized.

### Example 6

### USP14 is Associated with the 26S Proteasome

Comigration of EL-4 p58 with yeast Ubp6p SDS-PAGE was observed. Moreover, [¹²⁵I]-UbVS labeling of both p58 and Ubp6p is competed similarly by Ubal (FIG. 2B and 3B). As p58 may be the mammalian homolog of Ubp6, USP14 (Yin et al., Biochemistry, 39, 10001-10, 2000) polyclonal antibodies against synthetic peptides corresponding to USP14 were used herein.

In an immunoblot, these antisera detected a single polypeptide of a molecular weight consistent with that of USP14. The antiserum immunoprecipitated an [¹²⁵I]-UbVS-labeled polypeptide that comigrates with the 66 kDa [¹²⁵I]-UbVS labeled DUB observed in anti-20S IPs (FIG. 4B), suggesting that USP14 is associated with the proteasome. The observed difference in the USP14 to p37 ratio in whole lysates versus the immunoprecipitated samples is likely due to the fact that not all of the protein present in the lysate is proteasome associated, and also due to differences in the affinity of p37/USP14 for the proteasome.

Since the anti-USP14 anti-serum recognizes only SDS-denatured protein, direct coimmunoprecipitation of the proteasome with USP14 with antibodies directed against USP14 was not be possible. To confirm association of USP14 with the proteasome, Superose 6 fractions were labeled with [¹²⁵I]-UbVS, and the 20S complex was immunoprecipitated with antibodies against the 20S core. Parallel samples were immunoblotted for different proteasome subunits (FIG. 4C).

The data show that [¹²⁵I]-UbVS modified USP14 was found in the fractions corresponding to 26S proteasome complex (fractions 18-22), and was not found in fractions containing free 20S proteasomes (fractions 24-28). These results establish that USP14 is physically associated with the 26S proteasome complex.

### Example 7

### [¹²⁵I]-UbVS Labeling of USP14 is Increased upon Proteasome Inhibition

Complete Ub removal is thought to precede proteasomal proteolysis. Conversely, when proteasomal proteolysis is blocked, the resultant accumulation of Ub-conjugated substrates may elicit enhanced activity of DUBs. Thus the activities of the proteasome and associated DUBs may be interdependent.

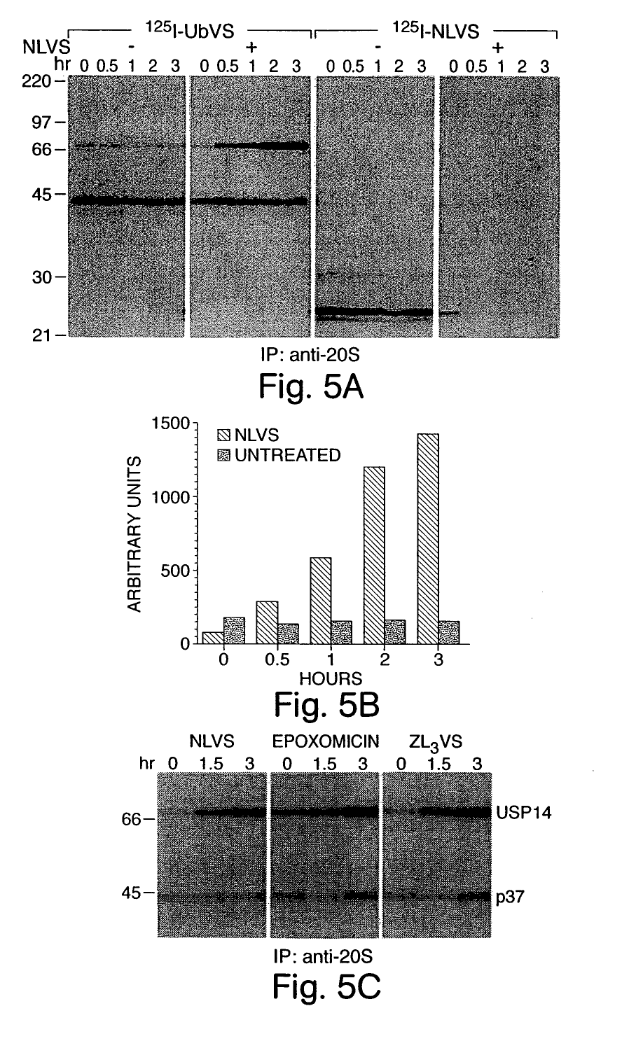

Vinyl sulfones are mechanism-based inhibitors, and consequently the increase of labeling of a given target enzyme is directly proportional to that enzyme's ability to bind and hydrolyse the peptide bond at the C-terminus of Ub. [¹²⁵I]-UbVS thus provides a convenient tool to examine the enzymatic activity of DUBs in response to proteasome inhibition. Intact EL4 cells were incubated with 50 μM NLVS for different times, and cell extracts were prepared. After incubation of the extracts with [¹²⁵I]-UbVS or [¹²⁵I]-NLVS, the proteasome was immunoprecipitated with an anti-20S serum (FIG. 5A).

Two [¹²⁵I]-UbVS labeled polypeptides were observed, a 45 kDa polypeptide (shown herein to be p37) and a 66 kDa polypeptide (shown herein to be USP14). Labeling of p37 did not change upon NLVS treatment (FIG. 5A, panel 2). In contrast, labeling of proteasome associated USP14 was found at a level which was increased up to 15-fold (FIG. 5B) upon inclusion of NLVS (FIG. 5A, panel 2), in a time-dependent manner. The observed increase in [¹²⁵I]-UbVS labeling of USP14 is consistent with observations of proteasome inhibition by NLVS, and treatment of cells with other proteasme inhibitors such as epoxomicin (Meng et al., Proc Natl Acad Sci USA, 96, 10403-8, 1999), ZL₃VS and lactacystin (Fenteany et al., Science, 268, 726-31, 1995) produces a similar effect (FIG. 5C). This increase may be due to recruitment of more USP14 to the proteasome complex, or could be caused by increased activity of USP14 already associated with the proteasome.

### Example 8

### Activation of USP14 in Response to Proteasome Inhibition

The observed increase in USP14 labeling is independent of protein synthesis, as inclusion of the protein synthesis inhibitor puromycin did not abolish increased USP14 labeling.

To determine whether pre-existing free USP14 is recruited to the proteasome in response to NLVS treatment, El-4 cells were incubated with NLVS, and subcellular fractionation of cell extracts was performed and carried out. The fractions were labeled with [¹²⁵I]-UbVS and analyzed by SDS-PAGE.

Both USP14 and p37 were found to be abundantly present in the 5 hr pellet fraction, which was enriched for proteasomes and other large subcellular particles, while most other DUBs remained cytosolic or were distributed over both cytosol and the 5 hr pellet (FIG. 6A). Analysis of the same fractions by immunoblot demonstrates that USP14 indeed co-sediments with Mss1, an ATPase in the 19S cap of the proteasome (FIG. 6B). As assessed by immunoblot, the protein levels of USP14 are comparable in cells treated with NLVS either before (FIG. 6B) or after lysis (FIG. 6C) and in control untreated cells, while the [¹²⁵I]-UbVS labeling of USP14 in NLVS treated 5 hr pellets and 1 hr supernatants is increased by two to three fold (average of 4 experiments, (FIG. 6C). The similar increase in USP14 labeling observed when EL-4 subcellular fractions were treated with NLVS after lysis (FIG. 6C) confirms lack of a requirement for ongoing protein synthesis. The 5 hr pellet fraction contained only a small amount of soluble USP14. The data show that recruitment of this material to the proteasome upon NLVS treatment would not account for the observed increase in labeling (FIG. 6C).

These surprising observations show that the increase in [¹²⁵I]-UbVS labeling of proteasome-associated USP14 is due to an increase in activity of USP14, and not due to recruitment of more USP14 to the proteasome complex. The increase in intensity of labeling is less pronounced in the 5 hr pellets than in anti-20S immunoprecipitations (FIG. 5A, B and 6A). This may be due to the presence of USP14 associated with other high molecular weight complexes in the 5 hr pellets, which are unaffected by the presence of a proteasome inhibitor or by the technical differences between the two methods, immunoisolation and subcellular fractionation, used to purify or enrich proteasomes. Immunoisolation does not result in quantitative recovery of proteasomes and may yield only a subset of the total proteasome population.

### Example 9

### The synthesis of Irreversible Inhibitors Equipped with a C-terminal Adenylate

The synthesis of irreversible inhibitors equipped with a C-terminal adenylate is being developed as well. The latter peptides should target proteins at the very beginning of Ub/Ubl-processing cycle, as the activation of Ubl's is likely to be analogous to the activation of Ub via the formation of an Ub-adenylate by the so-called El enzyme.

### Example 10

### Identification of Labeling Specificities of HAUb Derivatives

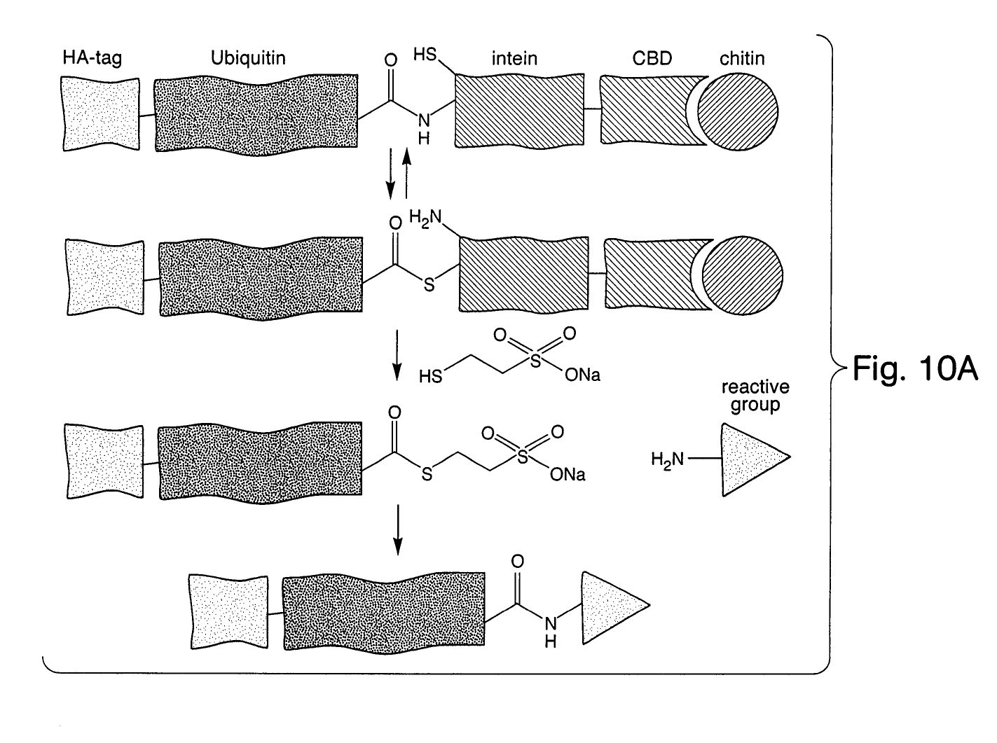

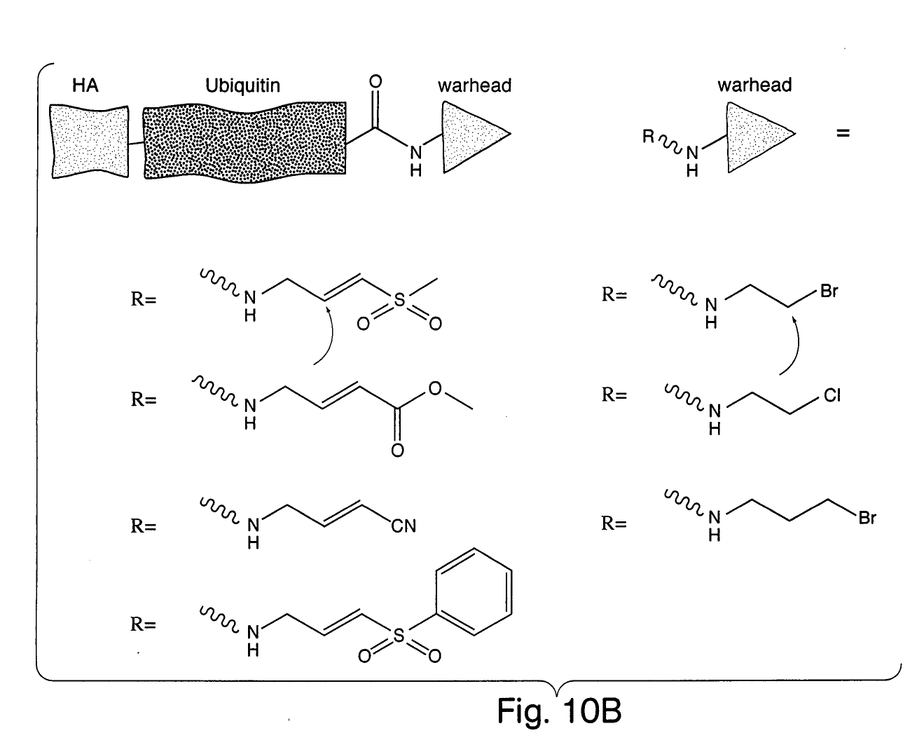

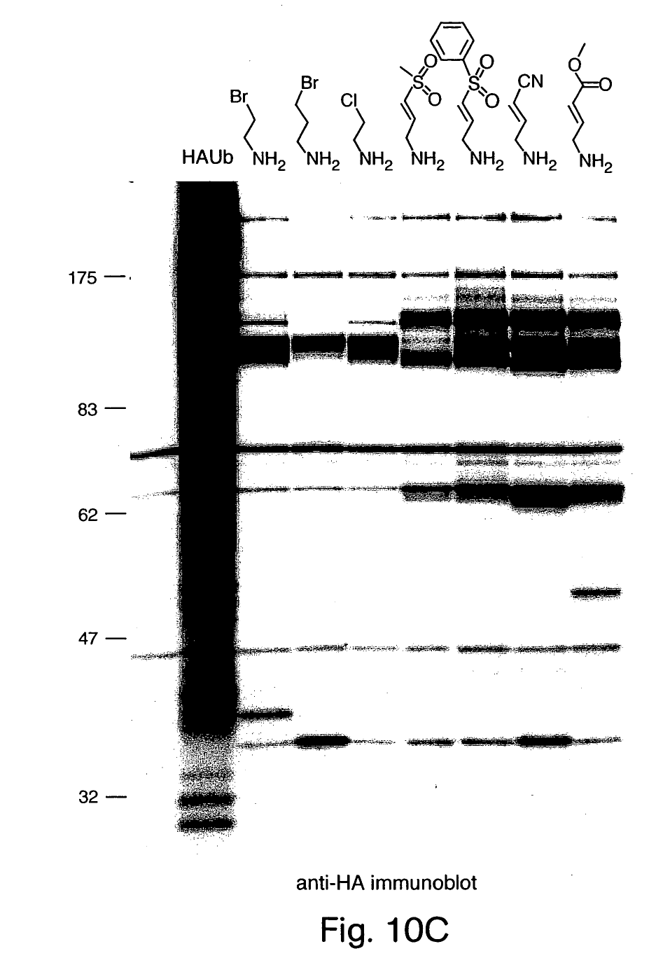

The proteome of a cell can be partially resolved by identifying subsets of proteins that interact with a set of inhibitors, providing the inhibitors can be visualized by an anlytical technique. The vectors herein are constructed as shown in FIGS. 10A and 10B, by a combination of in vivo recombinant fusion technology and expression, and chemical synthesis. The compositions provided herein, when mixed with a sample of the entire set of proteins in a cell lysate, interact selectively with certain proteins, as shown in FIGS. 7-9.

SDS-PAGE analysis with anti-hemagglutin (anti-HA) antibody blot (Western; FIG. 10C) revealed protein bands fractionated on the basis of molecular weight which were labeled by vectors derived from vector HAUb. Cells of strain EL-4 contain proteins capable of interacting specifically with the warheads on HAUBVS, HAUbVMe, HAUbVSPh, and HAUbBr1, HAUbCN, HAUbCl, and HAUbBr3, in contrast with control HAUb having the same vector having no reactive group or “warhead”. Proteins labeled with the vectors were resolved by 8% reducing SDS-PAGE and immunoblotted with anti-HA antibody to reveal the bands shown in each lane.

In the absence of a warhead, the vector was conjugated into poly-ubiquitin chains, producing a smear throughout the lane (see lane labeled HAUb). The presence however of the warhead restricted interaction of the vector to many fewer proteins, as shown by dark bands which have been stained by anti-HA antibody, and a low background.

The warhead terminating in the structure —(CH₂)₃Br (fourth lane from left) interacted with fewer proteins that a warhead terminating in the structure —CH₂(CH)₂CN. Further, warheads —(CH₂)₂Br and CH₂)₂Cl interacted with substantially the same proteins, however the former warhead also interacted with a protein of about 40K which was not seen to interact with the latter warhead. The warhead —CH₂(CH)₂SOme₂ failed to interact with a major band that is shown to interact with a similar warhead additionally having a phenyl group adduct. Identification of the proteins in these bands will yield results concerning similar features of interacting proteins.

### Example 11

### Purification and Sequence Analysis of Targeted Enzymes using Novel Semisynthetic Probes

The epitope tags can be used for the purification of targeted enzymes by immunoprecipitation (FIG. 11). The thus purified enzyme-Ub/Ubl adducts can be either directly sequenced by tandem mass spectrometry, or be sequenced by tandem mass spectrometry after (isoelectric-point focusing)SDS-PAGE (2D gel electrophoresis) followed by in-gel tryptic digest, leading to the identification enzymes involved in Ubl conjugation and deconjugation, a yet poorly characterized protein family. Examples of enzymes that were identified are given in FIG. 12 and Tables 3 and 4.

FIG. 11 shows proteins bound by different vectors bearing different reactive moities, i.e., different inhibitors, the proteins here analyzed by gel electrophoresis under non-denaturing conditions, as visualized by silver stain. Open circles indicate 19S cap bound USPs and open circles the 19S cap subunits.

These data show that the extent of purity and recovery of a particular Ub/Ubl component is specific for each of the HAUb vectors after immunoprecipitation, as can be determined by the methods and compositions provided herein. Tables 3 and 4 show the identity of labeled polypeptides from a HAUBVS-treated, nondenatured sample obtained herein, with their accession numbers. Examples with various cancer cells are shown herein (infra).

### Example 12

### Semisynthetic Synthesis of Inhibitors of Ubiquitin-Like Proteins

The method of synthesizing probes comprising amino acid fusion proteins and organic chemistry techniques was applied to the semisynthetic synthesis of specific inhibitors of enzymes involved in the processing of Ubl proteins. The synthesis of probe derivatives of the following Ubl proteins, APG8, APG12, UCRP, SUMO-1, NEDD-8, HUB1, URM-1, FAT10 and Fau were pursued.

Table 5 shows characteristics for these modifiers from the mouse. The amino acid sequences used were derived from clones of the proteins. The dash in the C-terminal sequence indicates the position where processing occurs, and were used to generate the mature Ub-like modifier. URM1, FAT10 and Apg12 were expressed in their mature form. The C-terminal sequence of Fau extends beyond the point shown.

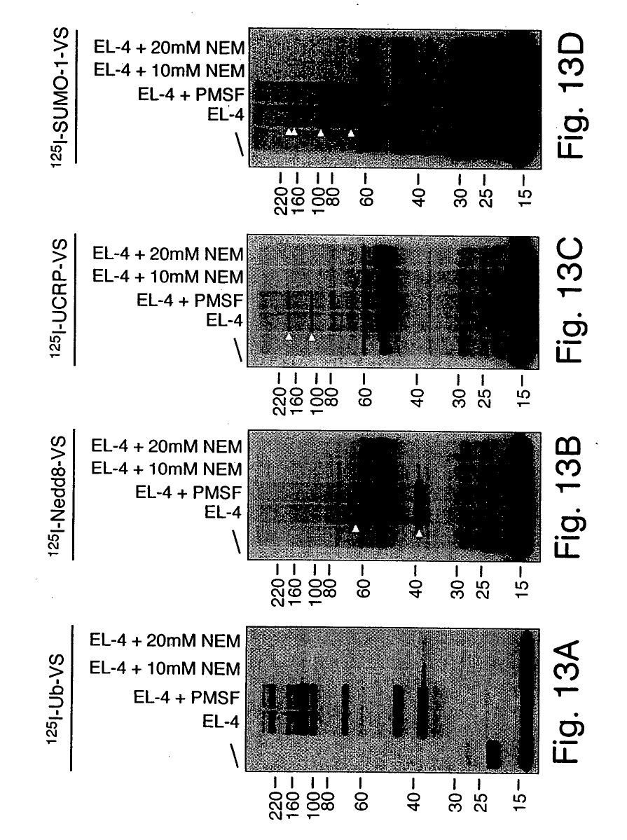

Modification of specific enzymes by Ub-like derivatives is shown in FIG. 13. Each protein-based probe generated a specific profile.

### Example 13

### Semisynthetic Synthesis of Inhibitors of Proteins Unrelated to Ubiquitin

The method is applicable to an even wider array of proteins, including those not necessarily related to Ub. The chemical reactive group (i.e. warhead) can be fine-tuned to direct reactivity to specific families of enzymes, by making it either more or less reactive.

### Example 14

### Development of Cell Permeable Derivatives and In Vivo Uses

A further application of these inhibitors is the development of cell permeable versions, for example, having an N-terminal TAT protein sequence, which will allow the inhibition of specific enzymes within the cell, greatly facilitating the investigation of the biological function of Ub and Ubl proteins. Protein-based semisynthetic probes described herein can also be introduced by micro-injection.

The newly developed inhibitors facilitate investigation of the biological function of Ub and Ubl pathways in health and disease. Apart from the identification of the relevant enzymes, the inhibitors, which are mechanism-based site affinity probes, can be used to compare the enzymatic activity in different (tissue) samples and disease states. Furthermore, the inhibitors will allow modulation of the enzymatic pathways, and thereby of the biological processes and disorders they are involved in.

### Example 15

### Identification of a Novel Ovarian Tumor Family Protein using HAUb Probes

Table 3 identifies a novel protein which was detected herein in mouse EL4 cells using the vector HAUbBr. The molecular weight was observed to be between 35 and 42 kDa (see Table 3, last line HSPC263 (OTU-protease). The novel DUB was found by its sequence to be a member of the ovarian tumor cell domain (OTU) family, and its identification herein is an example of an application of the methodology to isolation of novel proteins, as is its functional identification as a deubiquitinating enzyme.

It was herein further characterized by tandem mass spectometry (MS). The protein was previously known only as a fragment rather than as a complete protein, and was listed in databases as HSPC263 (human), having accession number is Q9P0B8 and the following amino acid sequence (SEQ ID NO: 5) using the one letter code for amino acids:

GCLKMAAEEPQQQKQEPLGSDSEGVNCLAYDEAIMAQQDRIQQEIAVQNP
LVSERLELSVLYKEYAEDDNIYQQKIKDLHKKYSYIRKTRPDGNCFYRAF
GFS HLEALLDDSKELQRFKAVSAKSKEDLVSQGFTEFTIEDFHNTFMDL
IEQVEKQTSVADLLASFNDQSTSDYLVVYLRLLTSGYLQRESKFFEHFIE
GGRTVKEFCQQEVEPMCKESDHIHIIALAQALSVSIQVEYMDRGEGGTTN
PHIFPEGSEPKVYLLYRPGHYDILYK

As the sequence was previously considered a fragment, and as data herein such as mobility on SDS-PAGE indicates that this sequence is the complete protein, and that the function is involved in deubiquination, these data both indicate that the protein is complete, and the nature of its function.

### Example 16

### USP Activity in B Cell Malignancies

An HAUb probe HAUbVME was used to examine proteins of cells from several multiple myeloma and several Burkitt's lymphoma cell lines, which were compared to proteins in LCL cells. All B cell malignant cell lines show bands not found in EL-4 mouse thymoma cells.

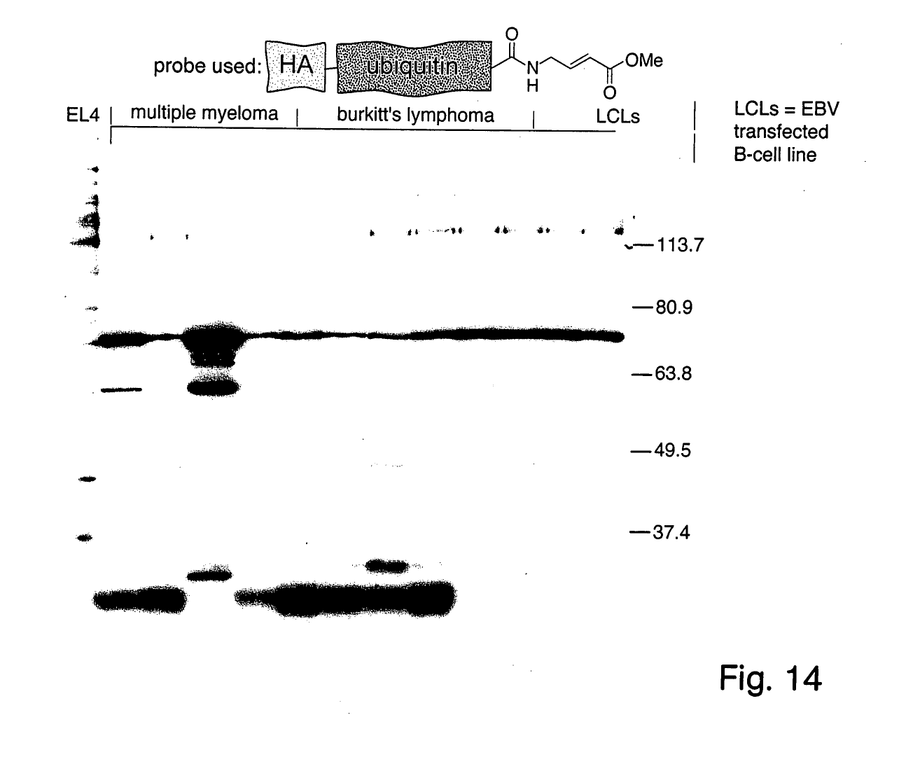

As shown in FIG. 14, between one to seven additional bands, or bands that differed in expression in the malignancy cell lines, compared to the LCL and EL-4 control extracts, were observed All malignant cell lines showed upregulated DUBs, UCH-L1 being the most prominent one. In the EL-4 cell control, all band were previously identified herein.

### Example 17

### Labeling Patterns Observed Following Stimulation of Human B Cells with Mitogens

Human B cells were probed with HAUbVME prior to treatment, and at one, 3 and 5 days following treatment with each of mitogens phytohemagglutinins L or M, or pokeweed mitogen, and were compared to probes of untreated cells. Substantial induction of high molecular weight protein bands specific to the probe were observed at 3 and 5 days with each of phytohemagglutinins L and M, however loss of expression of high molecular weight proteins was observed following pokeweed mitogen stimulation.

### Example 18

### Tetracycline-Induced Knockout of Transcription Factor YY1 Affects USP Translation

At four days after induction, probe of cells with HAUbVME shows loss of the 47.5 kDa protein band that was initially present. Increased expression of a very high molecular weight band, and decreased expression of another high molecular weight band were also observed.

### Example 19

### Use of Semisynthetic Probes for Fraction Identification during Purification

A useful application of the probes provided herein is for identification of positive fractions during standard biochemical purification, for example, of a de-ubiquitin enzymes. A crude cell extract was fractioned using a ubiquitin-sepharose column, followed by gel-filtration. During these procedures, the active DUBs were identified by blot using the probe.

This result shows that the probes herein can be used during isolating proteins, by identifying functional DUBs in crude cell lysates.

TABLE 1
Deubiquinating proteins and relationship to disease conditions

| Protein/Gene | Condition/Function |
|---|---|
| Ubiquitin Ligases | |
| E6AP (E3) | HPV infection (p53 |
| | degradation), Angelman |
| | syndrome |
| Cb1 (E3) | Oncogenic |
| BRCA1/BARD1(E3) | Fanconi anemia, breast cancer |
| Skp2(E3) | Human cancer/p27 |
| | degradation |
| Mdm2(E3) | P53 degradation |
| pVHL(E3) | Von Hippel Lindau syndrome |
| ICP0(E3) | HSV infection |
| Nedd4(E3) | Liddle's syndrome |
| UBE1L(E1-like) | Retinoid target triggering |
| | PML/RAR□ deg |
| HR6B(E2) | Sterility (mouse) |
| Itch(E3) | Immune dysregulation |
| | (mouse) |
| SCF Complex(E3) | NF-□B specific E3 |
| Parkin(E3) | Parkinson's disease |
| MID1(E3) | Opitz syndrome |
| Ubiquitin Specific Proteases | |
| (DUBs) | |
| UCHL1 | Parkinson's disease, GAD |
| | mouse |
| Unp | Oncogene |
| Usp6 (Tre2) | Oncogene |
| Usp7 (HAUSP) | Stabilizes p53, HSV infection |
| Usp8 (p85□/HUMORF8) | Oncogene |
| Usp9Y | Infertility |
| Usp14 | Ataxia mouse |
| CYLD | Familial cylindromatosis |
| BAP1 | BRCA associated protein, |
| | possible tumor suppressor |
| VDU1, 2 | Associated with and substrates |
| | for pVHL |
| Adenovirus protease | Adenoviral infection |

TABLE 2
Deubiquitinating enzymes of S. cerevisiae.

| Gene | MW | UbVS labeling | Characterization |
|---|---|---|---|
| Ubp1 | 93 | ++ | known Ubp |
| Ubp2 | 146 | ++ | known Ubp |
| Ubp3 | 110 | − | known Ubp |
| Ubp4/Doa4 | 105 | − | known Ubp |
| Ubp5 | 92 | − | known Ubp |
| Ubp6 | 57 | ++ | known Ubp |
| Ubp7 | 123 | − | putative |
| Ubp8 | 54 | − | putative |
| Ubp9 | 86 | − | putative |
| Ubp10/Dot4 | 88 | − | known Ubp |
| Ubp11 | 83 | − | knownUbp |
| Ubp12 | 143 | + | putative |
| Ubp13 | 77 | ND | putative |
| Ubp14 | 91 | − | known Ubp |
| Ubp15 | 143 | ++ | known Ubp |
| Ubp16 | 57 | ND | putative |
| Yuh1 | 26 | ++ | known Uch |

Labeling with ¹²⁵I-UbVS was determined as described in FIG. 3,
“++” indicates strong labeling,
“+” weak labeling, “−” no labeling,
ND - not determined.
The molecular weights of DUBs are based on the predicted sizes listed in the YPD database (http://www.proteome.com/databases/index.html).

TABLE 3
Enzymes Modified by HAUb-Based Probes

| | Accession | Predicted MW | | Number | | |
|---|---|---|---|---|---|---|
| Protein | Number | (kDa) | Observed MW | of Matches | Sequence Coverage (%) | Denatured |
| USP4 (Unp) | P35123 | 108 | 120, 130, 140 | 14 | 14 | + |
| USP5 (IsoT1) | P56399 | 95.8 | 120, 130, 140, | 18 | 24.3 | + |
| | | | 170, 220 | | | |
| USP7 (HAUSP) | Q93009 (h) | 128 | 140, 150, 170 | 11 | 12.2 | + |
| USP8 (Ubp-Y) | O9EQU1 | 122.5 | 156 | 10 | 30.2 | + |
| USP9X (FAFX, FAM) | P70398 | 290 | 300 | 22 | 11 | + |
| USP10 | P52479 | 87 | 120, 140, 150 | 4 | 5.1 | + |
| USP11 | P51784 (h) | 79 | 130 | 3 | 5 | + |
| USP12 (UBH1) | Q9O9M2 | 41 | 39, 50 | 1 | 3.7 | + |
| USP13 (IsoT3) | Q92995 (h) | 97.3 | 120 | 3 | 2.9 | + |
| USP14 (TGT) | Q9JMA1 | 56 | 62, 69, 75, 79 | 6 | 12.8 | + |
| USP15 | Q9Y4E8 (h) | 103 | 120, 130, 140 | 12 | 14.4 | + |
| USP15i | Q9Y6B6 (h) | 112 | 140, 130 | 6 | 6.4 | + |
| USP16 (Ubp-M) | Q99LG0 | 93 | 140 | 2 | 2.7 | + |
| USP19 | O94966 (h) | 151 | 120, 170, 200 | 5 | 6.3 | + |
| USP24 | Q9UPU5 (h) | 112 | 300? | 6 | 5.4 | + |
| USP25 | P57080 | 121 | | 9 | 13.7 | + |
| USP28 | Q95RU2 (h) | 122.5 | 160 | 3 | 3.2 | + |
| CYLD1 | Q96EHO (h) | 107 | 120 | 4 | 4.5 | + |
| m64E | Q98K76 | 147 | 170, 200 | 12 | 9.4 | + |
| USP frag 1 KIAA891 | Q96PZ6 | 87 (frag) | 140 | 2 | 5.5 | + |
| UCH-L1 | Q9R0P9 | 24.8 | 37 | 2 | 9.4 | + |
| UCH-L3 | Q9JKB1 | 26 | 31, 39, 41 | 11 | 57 | + |
| UCH37 | Q9WUP7 | 37.6 | 39, 48, 56 | 4 | 13.1 | + |
| HSPC263 (OTU-protease) | Q9P0B8 | 31.6 (frag) | 42, 35 (VS, | 4 | 16.2 | + |
| | | | unmodified) | | | |

| | | Ub Derivative | Activity | |
|---|---|---|---|---|
| Protein | Nondenatured | Modified by | Demonstrated | Remarks |
| USP4 (Unp) | + | VS, VME, Br2 | [67] | binds pRb, p107, p130 [38] |
| USP5 (IsoT1) | + | VS, VME, Br2 | [39] | disassembles free poly-Ub chains [39] |
| USP7 (HAUSP) | + | VS, VME, Br2 | [58] | binds ICP0 (HCMV), TRAF 1-5; binds |
| | | | | and deubiquitinates p63 [7, 40, 41] |
| USP8 (Ubp-Y) | + | VS, VME | [42] | binds Ras-GRF1, Hbp; levels increase |
| | | | | with growth stimulation [42-44] |
| USP9X (FAFX, FAM) | + | VS, VME, Br2 | [45] | Regulates B-catenin and AF-8, fly homolog |
| | | | | deubiquitinates liquid facets |
| | | | | [45, 46] |
| USP10 | + | VS, VME | [47] | G3BP binding inhibits activity [47] |
| USP11 | ND | VME | [48] | binds RanBPM [48] |
| USP12 (UBH1) | ND | VME | [59] | |
| USP13 (IsoT3) | ND | VME | this study | 64% Identical to USP5 [49] |
| USP14 (TGT) | + | VS, VME | [60] | Proteasome bound; activity modulated |
| | | | | by 268 association [14, 60] |
| USP15 | + | VS, VME, Br2 | [61] | 60% Identical to USP4 [51] |
| USP15i | + | VS, VME, Br2 | this stuady | splice variant of USP15 |
| USP16 (Ubp-M) | ND | VS, VME, Br2 | [62] | de-Ub histone H2A; binds chromatin; |
| | | | | phosphorylated during the cell |
| | | | | cycle [52] |
| USP19 | + | VS, VME, Br2 | this study | |
| USP24 | ND | VS, VME, Br2 | this study | |
| USP25 | ND | VME | this study | |
| USP28 | ND | VME | this study | |
| CYLD1 | ND | VME | this study | tumor suppressor gene mutated in cylindromatosis |
| | | | | [8] |
| m64E | + | VS, VME, Br2 | this study | mutant of fly homolog enhances position |
| | | | | effect variegation [53] |
| USP frag 1 KIAA891 | ND | VME | this study | fragment |
| UCH-L1 | + | VS, VME | [61] | linked to Parkinson's disease mutated |
| | | | | in gracile axonal dystrophy in mice |
| | | | | [8, 54] |
| UCH-L3 | + | VS, VME, Br2 | [61] | cleaves Ub-gene products KO mouse |
| | | | | has no phenotype [55, 56] |
| UCH37 | + | VS, VME | [22] | 108 cap subunit; edits poly-Ub chains |
| | | | | [21, 22] |
| HSPC263 (OTU-protease) | ND | Br2 | this study | An unmodified version is detected in |
| | | | | non-denatured HAUbVS immunoprecipitations |

TABLE 4
Proteins That May Associate with DUBs

| | Accession | Predicted MW | Observed MW | Number of | Sequence | |
|---|---|---|---|---|---|---|
| Protein | Number | (kDa) | (kDa) | Matches | Coverage (%) | Remarks |
| S1 (Rpn2) | Q99460 (h) | 106 | 115 | 3 | 6.2 | 19S cap subunit (base) |
| S2 (Rpn1) | Q13200 (h) | 100 | 97 | 18 | 26.2 | 19S cap subunit (base) |
| S3 (Rpn3) | P14685 | 60.7 | 61 | 16 | 33.2 | 19S cap subunit (lid) |
| S4 (Rpt2) | Q03527 (h) | 49 | 59 | 12 | 33.6 | 19S cap subunit (base) |
| S7 (Rpt1) | P46471 | 48.5 | 48 | 4 | 10.2 | 19S cap subunit (base) |
| S9 (Rpn6) | Q00495 (h) | 47.4 | 48 | 1 | 2.8 | 19S cap subunit (lid) |
| S10B (Rpt4) | Q92524 (h) | 44 | 43, 44 | 8 | 26.7 | 19S cap subunit (base) |
| S10A (Rpn7) | Q99JI4 | 45.5 | 44 | 20 | 47.3 | 19S cap subunit (lid) |
| S11 (Rpn9) | Q9WVJ2 | 42.8 | 41 | 16 | 43.4 | 19S cap subunit (lid) |
| S12 (Rpn8) | P26516 | 36.5 | 39 | 3 | 15 | 19S cap subunit (lid) |
| S13 (Rpn11) | O35593 | 34.5 | 34 | 1 | 4.2 | 19S cap subunit (lid) |
| DNA methyltransferase | P13864 | 183 | 200 | 5 | 16.4 | Dmnt1 |
| DNA pol1 subunit | CAC96831 | 98.5 | 115 | 1 | 1.1 | |
| RNA helicase A | O70133 | 149 | 150 | 10 | 8 | binds mRNa^{a} |
| RNAbp EWS | Q01844 | 68 | 75 | 1 | 2.1 | binds mRNA^{a} |
| PolyA-BP | P11940 | 70 | 69 | 13 | 24.5 | binds mRNA^{a} |
| RNA helicase PL10 | P16381 | 73 | 73 | 2 | 3.9 | binds mRNA^{a} |
| thioredoxin-like | CAC40691 | 37 | 34 | 1 | 3.9 | |
| aminotransferase | Q98JR5 | 44 | 39 | 1 | 2 | |

TABLE 5
Ubiquitin-like modifiers to which UBL-intein-CBD expression approach was applied

| | | % identity | | | |
|---|---|---|---|---|---|
| Ubiquitin-like | % identity | mouse | | UBL-intein-CBD | Radio- |
| modifier | to ubiquitin | vs. human | C-terminal amino acids | solubility | iodination |
| Ubiquitin | 100 | 100 | LVLRLRGG-XXXXX | + | + |
| Nedd8 | 57 | 100 | LVLALRGG-GGLGQ | + | + |
| UCRP | 28/34 | 65 | KHLRLRGG-GGDQCA | + | + |
| SUMO-1 | 18 | 100 | VYQEQTGG-HSTV | + | + |
| URM-1 | 13 | 93 | FISTLHGG | + | + |
| Fau | 31 | 95 | VAGRMLGG-KVHGSLARAGKV | + | N.D. |
| FAT-10 | 28/34 | 68 | LTTHCTGG | − | N.D. |
| HUB1 | 22 | 100 | GMNLELYY-Q | − | N.D. |
| Apg12 | 11 | 89 | YCKSQAWG | − | N.D. |

Characteristics are shown for Ub-like modifiers from the mouse.
The amino acid sequences used are derived from the clones described in the experimental procedures section.
Percentages identity apply to the processed forms of the Ub-like modifiers.
The dash in the C-terminal sequence indicates the position where processing occurs to generate the mature Ub-like modifier.
URM1, FAT10 and Apg12 are expressed in their mature form.
The C-terminal sequence of Fau extends beyond the point shown.
+: soluble or could be radio-iodinated,
−: insoluble,
N.D.: not done.

1. A fusion peptide comprising an amino acid sequence of components, in order from the amino terminus: an epitope label; an amino acid sequence of a ubiquitin or ubiquitin -like protein; an intein; and a chitin binding protein.

2. A vector encoding the peptide of claim 1.

3. A semi-synthetic protein-based site directed probe for identification and inhibition of a class of genomic proteins, the probe comprising the epitope label and ubiquitin or ubiquitin like protein according to claim 1, and further comprising a potential inhibitory group at the carboxy terminus of the ubiquitin or ubiquitin -like protein, wherein the inhibitory group is specific for an enzymatic activity of a ubiquinating or a deubiquinating enzyme.

4. A site directed probe according to claim 3, wherein the group is reversibly inhibitory.

5. A site directed probe according to claim 4, wherein the reversible inhibitory group is an aldehyde or a boronate.

6. A site directed probe according to claim 3, wherein the group is irreversibly inhibitory.

7. A site directed probe according to claim 6, wherein the irreversibly inhibitory group is an electron with drawing group.

8. A site directed probe according to claim 6, wherein the irreversibly inhibitory group is a Michael acceptor-containing group or an alkylating group.

9. A site directed probe according to claim 8, wherein the Michael acceptor-containing group is selected from compounds 3-vinylmethylsulfone; 3-vinylphenylsulfone; 3-vinylnitrile; and 2-carboxyvinylmethane.

10. A method of obtaining a semi-synthetic protein-based site directed probe for identification and inhibition of a subset of a proteome, the method comprising:
providing a a fusion protein encoded by a nucleic acid vector, the fusion protein having: an epitope tag, a domain having an amino acid sequence from a member protein of the subset, an intein, and an affinity creating binding peptide;
breaking a peptide bond located between the domain and the intein, to yield a thioester at the carboxy terminus of the representative peptide; and
further reacting the thioester to yield an active reversible aldehyde or an electron withdrawing group at the carboxyterminus of the domain, thereby obtaining the site directed probe.

11. A method according to claim 10, wherein the member protein is a ubiquitin or a ubiquitin-like protein.

12. A method according to claim 10, wherein the member protein is selected from the group consisting of UCH-L3, APG8, APG12, UCRP, SUMO-1. NEDD-8, HUB1, URM-1, FAT10 and Fau.

13. A method according to claim 10, wherein the epitope tag is selected from hemagglutinin, Flag, Myc and His₆.

14. A method according to claim 10, wherein prior to breaking the peptide bond between the intein and the domain, the method further comprises purifying the fusion protein by contacting a preparation comprising the fusion protein the with an immobilized binding partner of the affinity binding peptide.

15. A method according to claim 14, wherein the affinity binding domain is a chitin binding domain, and the immobilized binding partner is immobilized chitin.

16. A probe according to any of the methods of claims 10-15.

17. A method for obtaining a semi-synthetic protein-based site directed probe for identification and inhibition of ubiquitin and ubiquitin-like proteins, the method comprising:
providing a fusion protein encoded by a nucleic acid vector, the fusion protein having: an hemagglutinin tag, a domain from a ubiquitin or a ubiquitin-like protein, an intein, and a chitin binding peptide;
breaking a peptide bond located between the domain and the intein, to yield a thioester at the carboxy terminus of the representative; and
further reacting the thioester to yield an active reversible aldehyde or an electron withdrawing group at the carboxyterminus of the domain, thereby obtaining the site directed probe.

18. A probe obtained by the method of claim 17, wherein the ubiquitin-like protein is selected from the group consisting of UCH-L3, APG8, APG12, UCRP, SUMO-1, NEDD-8, HUB1, URM-1, FAT10 and Fau.

19. A method of identifying a subset of a proteome, wherein members of the subset share a functional pathway, the method comprising:
preparing a semi-synthetic protein-based site directed probe having an amino acid sequence comprising an epitope tag, a peptide from a member of the subset having an enzyme activity, and an inhibitory group at the carboxy terminus of the member peptide, the inhibitory group having ability to contact and inhibit an activity of the enzyme;
contacting a lysate of the cell with the probe; and
analyzing the lysate by reducing SDS gel electrophesis and immunoblotting with an antibody specific for the tag, such that cell lysate components encoded by the members of the subset bind to the probe, and react with the antibody to visualize bands on the gel, thereby identifying the subset of the proteome that share the pathway.

20. A method according to claim 19, wherein the pathway is ubiquitination or deubiquitination.

21. A protein identified according to the method of claim 19.

22. A deubiquitination protein having a sequence according to SEQ ID NO: 5.

23. The protein having amino acid sequence consisting substantially of GCLKMAAEEPQQQKQEPLGSDSEGVNCLAYDEAIMAQQDRIQQEIAVQNPL VSERLELSVLYKEYAEDDNIYQQKIKDLHKKYSYIRKTRPDGNCFYRAFGFS HLEALLDDSKELQRFKAVSAKSKEDLVSQGPTEFTIEDFHNTFMDLIEQVEK QTSVADLLASFNDQSTSDYLVVYLRLLTSGYLQRESKFFEHFIEGGRTVKEF CQQEVEPMCKESDHIHIIALAQALSVSIQVEYMDRGEGGTTNPHIFPEGSEP KVYLLYRPGHYDILYK.

24. A kit for identifying ubiquitination and deubiquitination proteins in a cell, comprising a semi-synthetic protein-based site directed probe having an epitope tag, an amino acid sequence of a ubiquitin or ubiquitin-like protein, and an inhibitory compound covalently bound to the carboxy terminus of the amino acid sequence, and instructions for use.

25. A kit according to claim 24, further comprising an antibody specific for the epitope tag.

26. A kit according to claim 24, further comprising reagents for lysing a cell.

27. A kit according to claim 26, further comprising reagents for fractionating cell components.

28. A kit for fractioning a cell lysate, comprising the components of the kit of claim 24, and an affinity chromatography material covalently attached to a ubiquitin or a ubiquitin-like protein or fragment of a ubiquitin-like protein.

29. A kit according to claim 24, wherein the ubiquitin-like protein is selected from the group of: UCH-L3, APG8, APG12, UCRP, SUMO-1, NEDD-8, HUB1, URM-1, FAT10 and Fau.

30. A kit for diagnosis of a disease comprising a semi-synthetic protein-based site directed probe according to claim 24.
